SEQUENCE LISTING (9 sequences)

SEQ ID NO: 1 - RNA, 1434 nt, Mogibacterium sp.
  source 1..1434: mol_type = rRNA
gatgaacgctggcggcgtgcctaatacatgcaagtcgagcgagaagcttggaaatgacgcttcggttgaatttccaagcggacagcggcggacgggtgagtaacgcgtaggcaacctgcccctgtacagagggatagccattggaaacgatgattaaaacctcatgacaccgtagtagcacatgctacatcggtcaaagatttatcggtcagggatgggcctccgtctgattaactggttggtgaggtaacggctcaccaaggtgacgatcagtagccgacctgagagggtgatcggccacattggaactgagacacggtccaaacttctacggaaggcagcagtagggaatcttgcacaatgggcgtaagcctgatgcagcaacgccgcgtgaaggatgaaggcttcgggttgtaaacttctgttctaagggaagaaagaaatgacggtaccttaggagcaagccccggctaactacgtgccagcagccgcggtaatacgtagggggcaagcgttatccggaattattgggcgtaaagagtgcgtaggtggttacctaagcgcaaggtttaaattagaggctcaacctctacatgccttgcgaactgggctacttgagtgcaggaggggaaagcggaattcctagtgtagcggtgaaatgcgtagatattaggaggaacaccggcggcgaaggcggctttctggactgtaactgacactgaggcacgaaagcgtgggtagcaaacaggattagataccctggtagtccacgccgtaaacgatgagcactaggtgttgggtccgttaggactcagtgccgcagttaacgcaataagtgtccgcctgggagtacgctcgcaagagtaaactcaaggaattgacgggcacccgcacaagcaggggagcttgtggtttaattcgaagcaacgcgaagaaccttaccagggcttgacatcctgctgacagaaccttaatcggttttttcttcggacagcagagacaggtggtgcatggttgtcgtcagctcgtgtcgtgagatgttgggttaagtcccgcaacgagcgcaacccttgtcgctagttactaacattcagttgaggactctagcgagactgccgaggtcaactcggaggaaggtggggatgacgtcaaatcatcatgccccttatgttctgggctacacacgtgctacaatggtcggtacaatgagatgcaatactgcgaagtggagcgaaacaccaaaaccgatcccagttcggattgtaggctgcaactcgcctacatgaagtcggagttgctagtaatcgcagatcagaatgctgcggtgaatgcgttcccgggtcttgtacacaccgcccgtcacaccatggaagttgggggtgcccaaagtcggttaattaatctatcgcctaaggcaaaaccaatgactggggt

SEQ ID NO: 2 - RNA, 1406 nt, Eubacterium sp.
  source 1..1406: mol_type = rRNA
gtgctgcagagaagagtttgatcctggctcaggatgaacgctggcggcgtgcctaacacatgcaagttgagcgagaaatcacttaaagaagcttcggtagacttaagagatggagagcagcggacgggtgaggaacgcgtgggaaacctgcccttgacaggaggatagccgagagaaatttcgatttaatacttcataaagcagagcattcgcatggatgaactgccaaagaattatcggtcaaggatggtccagcgtctgattagctggttggtaaggtaccggcttaccaaggcgacgatcagtagccggcctgagagggtgaacggccacattggaactgagacacggtccagactctacgggaggcagcagtggggaatattgcacaatgggcgaagcctgatgcagcaacgccgcgtgaaggaagaaggctttcgagtcgtaaacttctgtccaaagggaagaataatgacggtacctttgaagaaagccccggctaactacgtgccagcagccgcggtaatacgtagggggcgagcgttatccggaattactgggcgtaaagagtatgtaggtggttaagtaagcgtagggtttaaggcgacagcccaactgtcgtatgccccgcgagctgtttaacttgagtacaggaggggaaggcggaattcctagtgtagcgctgaaatgcgtagatattaggaggaacaccagtggcgaaggcggccttctggactgtaactgacactgagatacgaaagcgtgggtagcaaacaggattagataccctggtagtccacgccgtaaacgatgagcactaggtgtcgggctcgcaagagttcggtgccggagcaaacgcattaagtgctccgcctggggagtacgcacgcaagtgtgaaactcaaaggaattgacggggacccgcacaagcagcggagcatgtggtttaattcgaagcaacgcgaagaaccttaccaggacttgacatcccactgaaagctcggttaaaactgagcccttcttcggaacagtggagacaggtggtgcatggttgtcgtcagctcgtgtcgtgagatgttgggttaagtcccgcaacgagcgcaacccttgccgttagttgccatcattaagttgggcactctaatgggactgccggggagaacccggaggaaggcggggatgacgtcaaatcatcatgccccttatgttctgggctacacacgtgctacaatggccgtcacagagggaagcgagagagcgatcttaagcgaaaccaaaaaggcggtcccagttcggactgcaggctgcaactcgcctgcacgaagccggagttgctagtaatcgcggatcagaatgtcgcggtgaatgcgttcccgggtcttgtacacaccgccggtggatccgtg

SEQ ID NO: 3 - RNA, 1504 nt, Dialister sp.
  source 1..1504: mol_type = rRNA
cgaacgctggcggcgtgcttaacacatgcaagtcgaacggaaagagatgaaagagcttgctcttttattaatttcagtggcaaacgggtgagtaacacgtaaacaacctgccttaaggatggggataacagacggaaacgactgctaataccgaatacgttctaagcatcgcatggtgcatagaagaaagggtggcctctacaagaaagctatcgccttaagaggggtttgcgactgattaggtagttggtgaggtaacggctcaccaagccgacgatcagtagccggtctgagaggatgaacggccacactggaactgagacacggtccagactcctacgggaggcagcagtggggaatcttccgcaatggacgaaagtctgacggagcaacgccgcgtgaacgaagaaggtcttcggattgtaaagttctgtgattcgggacgaaagggtttgtggtgaataatcattgacattgacggtaccgaaaaagcaagccacggctaactacgtgccagcagccgcggtaatacgtaggtggcaagcgttgtccggaattattgggcgtaaagcgcgcgcaggcggtttcttaagtccatcttaaaagcgtggggctcaaccccatgaggggatggaaactgggaagctggagtatcggagaggaaagtggaattcctagtgtagcggtgaaatgcgtagagattaggaagaacaccggtggcgaaggcgactttctagacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaagccttgacattgatcgcaatccatagaaatatggagttcttcttcggaagacgagaaaacaggtggtgcacggctgtcgtcagctcgtgtcgtgagatgttgggttaagtcccgcaacgagcgcaacccctatttcctgttaccagcacgtaaaggtggggactcaggagagaccgccgcggacaacgcggaggaaggcggggatgacgtcaagtcatcatgccccttatggcttgggctacacacgtactacaatgggtgccaacaaagagaagcgaaatcgcgagatggagcggacctcataaacgcacccccagttcagattgcaggctgcaactcgcctgcatgaagtaggaatcgctagtaatcgcgggtcagcataccgcggtgaatacgttcccgggccttgtacacaccgcccgtcacactatgagagttagagacacccgaagccggtgaggtaaccgcaagggaccagccgtctaaggtggagctgatgattggagtgaagtcgtaacaag

SEQ ID NO: 4 - RNA, 1517 nt, Enterococcus sp.
  source 1..1517: mol_type = rRNA
agagtttgatcctggctcaggacgaacgctggcggcgtgcctaatacatgcaagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgacaagcggtggagcatgtgtttaattcgaagcaacgcgaagaaccttaccaggtcttgacatcctttgaccactctagagatagagctttcccttcggggacaaagtgacagtggtgcatggttgtcgtcagctcgtgtcgtgagatgttgggttaagtccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagccagccgcctaaggtgggatagatgatttgggtgaagtcgtaacaaggtaacc

SEQ ID NO: 5 - RNA, 1408 nt, Anaeroglobus sp.
  source 1..1408: mol_type = rRNA
cttacgatcgagtggcaaacgggtgagtaacgcgtaaacaacctgccccgcagatggggacaacagctggaaacggctgctaataccgaatacggtcctcttagcgcatggtaagaggaagaaagggtggcctctggaacaagctaccgctgtgggaggggtttgcgtctgattagctggttggaggggtaacggcccaccaaggcgacgatcagtagccggtctgagaggatgaacggccacattggaactgagacacggtccagactcctacgggaggcagcagtggggaatcttccgcaatgggcgaaagcctgacggagcaacgccgcgtgagtgaagacggccttcgggttgtaaagctctgttataggggacgaacggccgggtagcgaagaggtagccggcatgacggtaccgtaagagaaagccacggctaactacgtgccagcagccgcggtaatacgtaggtggcaagcgttgtccggaatgattgggcgtaaagggcgcgcaggcggctgtgtaagtctgtctaaaaagtgcggggctaaaccccgtgagaggatggaaactggacagctgagagtgtcggagaggaaagcggaattcctagtgtagcggtgaaatgcgtagatattaggaggaacaccggtggcgaaagcggctttctggacgacaactgacgctgaggcgcgaaagccaggggagcaaacgggattagataccccggtagtcctggccgtaaacgatgggtactaggtgtaggaggtatcgacccctcctgtgccggagttaacgcaataagtaccccgcctggggagtacggccgcaaggctgaaactcaaaggaattgacgggggcccgcacaagcggtggagtatgtggtttaattcgacgcaacgcgaagaaccttaccaagccttgacattgctcgcaacgggtagagatacctggttcttcttcggaagacgagacaacaggtggtgcacggctgtcgtcagctcgtgtcgtgagatgttgggttaagtcccgcaacgagcgcaacccctatcttcagttaccagcacgtagcggtggggactcaggagagacagccgcagacaatgcggaggaaggcggggacgacgtcaagtcatcatgccccttatggcttgggctacacacgtactacaatggctctaaatagagggaagcgaaggagcgatccggagcaaaccccgcaaacagagtcccagttcggattgcaggctgcaactcgcctgcatgaaggaggaatcgctagtaatcgcaggtcagcatactgcggtgaatacgttcccgggccttgtacacaccgcccgtcacaccacgaaagtcattcacacccgaagccggtgaggtaaccgcaaggagccagccgtcaaaggtg

SEQ ID NO: 6 - RNA, 1465 nt, Peptostreptococcus sp.
  source 1..1465: mol_type = rRNA
gatgaacgctggcggcgtgcctaacacatgcaagtcgagcgagggtttgctcagtattgagtattctaagactagaatgttcaattctgagcaaaaccaagcggcggacgggtgagtaacgcgtgggtaacctgccctatacacatggataacatactgaaaagtttactaatacatgataatatatatttgcggcatcgcagatatatcaaagtgttagcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgaaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagccgccgaaactggaggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgctgtaaacgatgagtactaggtgtcgggggttacccccctcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaagcttgacatccctcggacaggtgtttaatcacacccttccttcgggactgaggtgacaggtggtgcatggttgtcgtcagctcgtgtcgtgagatgttgggttaagtcccgcaacgagcgcaacccttgtctttagttgccagcattcagttgggcactctagagagactgccagggataacctggaggaaggtggggatgacgtcaaatcatcatgccccttatgcttagggctacacacgtgctacaatgggtggtacagagggttgccaaaccgtgaggtggagccaatcccttaaagccattctcagttcggattgtaggctgaaactcgcctacatgaagctggagttactagtaatcgcagatcagaatgctgcggtgaatgcgttcccgggtcttgtacacaccgcccgtcacaccacgggagtcggaaacacccgaagccgattatccaaccgcaaggaggaagtcgtcgaaggtggcgtcnataactggggtg

SEQ ID NO: 7 - RNA, 1487 nt, Solobacterium sp.
  source 1..1487: mol_type = rRNA
agagtttgatcctggctcagtatgaactctgccggcgtgcctaatacttgcaagtcgaacgctgaagatctagcttgctagatcgaaggagtgccgaacgggtgagtaatacataagcaacctacccacgaagactgggataatctctggaaacggggactaataccggataggtaatcggaaggcatcttctggttattaaaggttaaaaacactggtggatgggcttatggcgcattagttagttggtgaggtaacggcccaccaagacgatgatgcgtagccggcctgagagggtgaacggccacattgggactgagacacggcccagactctacgggaggcagcagtagggaattttcggcaataggggcaaccctgaccgagcaacgccgcgtgagtgaagacggccttcgggttgtaaaagctcttgtttgtaagggaagaacggtagatagagaatatctaagtgacggtaccttaccagaaagccacggctaactacgtgccagcagccgcggtaatacgtaggtggcgaccgttatccggaattattggccgtaaagggtgcgtaggcggcctgttaagtaagtggttaaattgttgggctcaacccaatccagccacttaaactggcaggctagagtattggagaggcaagtggaattccatgtgtagcggtaaaatgcgtagatatatggaggaacaccagtggcgaaggcggcttgctagccaaagactgacgctcatgcacgaaaccgtggggagccaataggattagataccctagtagtccacgccgtaaccgatgaatactaagtattggggaaactcagtgctgcactaacgcaataagtattccgcctgtggagtatgcacgcaagtgtgaaacataaaggaattgacgggggcccgcacaagcggtggagtatgtggtttaattcgacgcaacgcgaagaaccttaccaggccttgacatcccttgcaaagctgtagagatacagtagaggttatcaaggagacaggtggttgcatggtgtcgtcagctcgtgtcgtgagatgttgggttaagtccggcaacgagcgcaacccttgccttcagttaccagcatttagttggggactctggagggactgccggtgataaaccggaggaaggtggggatgacgtcaaatcatcatgccccttatggcctgggctacacacgtactacaatggctgttacaacgtgcagcgacctagcgataggaagcgaatcactaaaagacagtctcagttcggattgaagtctgcaactcgacttcatgaagctggaatcgctagtaatcgcggatcagaatgccgcggtgaatacgttctcgggccttgtacacaccgcccgtcataccatgagagctggtaatacccgaggccggtggcctaaccgcaaggaaggagccgtcgaaggtaggactagtgattggggtcaagtcgtaacaaggtaacc

SEQ ID NO: 8 - RNA, 1450 nt, Actinomyces sp.
  source 1..1450: mol_type = rRNA
tcctgactcaggacgaacgctgccggcgtacataacacatgcaagtcgaacggtgaaggggcctgcttttgtgggtcctggatgagtggcgaacgggtgagtaacacgtgagtaacctgcccccttcttctggataaccgcatgaaagtgtggctaatacgggatattctgggtctgtcgcatggtgggcctgggaaagattgcgccttttttggtgtttttggtgggggatgggctcgcggcctatcagcttgttggtggggtgatggcctgccaaggcggtgacgggtagccggcctgagagggtggacggtcacactgggactgagacacggcccagactcctacgggaggcagcagtggggaatattgcacaatgggcgcaagcctgatgcagcgacgccgcgtgagggatggaggccttcgggttgtgaacctctttcgccagtgaagcaggcctgcctcgtttgtgggtgggttgacggtagctggataagaagcgccggctaactacgtgccagcagccgcggtaatacgtagggcgcgagcgttgtccggaattattgggcgtaaagagctcgtaggcggctggtcgcgtctgtcgtgaaatcctctggcttaactgggggcttgcggtgggtacgggccggcttgagtgcggtaggggagactggaactcctggtgtagcggtggaatgcgcagatatcaggaagaacaccggtggcgaaggcgggtctctgggccgttactgacgctgaggagcgaaagcgtggggagcgaacaggattagataccctggtagtccacgccgtaaacgttgggcactaggtgtggggggccttttccgggtcttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgtgccggtctgctccggagacggggtttcctcctttgtgggggctggttcacaggtggtgcatggttgtcgtcagctcgtgtcgtgagatgttgggttaagtcccgcaacgagcgcaacccttgtctcgtgttgccagcacgttgtggtggggactcgcgggagactgccggggtcaactcggaggaaggtggggatgacgtcaaatcatcatgccccttatgtcttgggcttcacgcatgctacaatggccggtacagagggctgcgataccgtgaggtggagcgaatcccttaaagccggtctcagttcggatcggtgtctgcaactcgacaccgtgaagttggagtcgctagtaatcgcagatcagcaacgctgcggtgaatacgttctcgggccttgtacacaccgcccgtcacgtcatgaaagtcggcaacacccgaagcccgt

SEQ ID NO: 9 - RNA, 1446 nt, Alistipes sp.
  source 1..1446: mol_type = rRNA
gatgaacgctagcggcaggcttaacacatgcaagtcgaggggcagcacgaggtagcaatactttggtggcgaccggcgcacgggtgcgtaacgcgtatgcaacctacctttaacaggggcataacactgagaaattggtactaattccccataacattcgagaaggcatcttcttgggttaaaaactccggtggttaaagatgggcatgcgttgtattagctagttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtactagggtaaacgctcttacgtgtaggagcctgaaagtatagtacgaataaggatcggctaactccgtgccagcagccgcggtaatacggaggatccaagcgttatccggatttattgggtttaaagggtgcgtaggcggtttgataagttagaggtgaaataccggggctcaactccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagtgacgattctggaaacaggatttcccttcggggcacgaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggccgctaccccgcgaggggatgccaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggct

(19) United States
(12) Patent Application Publication
(10) Pub. No.: US 2024/0368693 A1
(43) Pub. Date: Nov. 7, 2024

(54) METHOD AND SYSTEM FOR RISK ASSESSMENT OF AUTISM SPECTRUM DISORDER IN A SUBJECT

(71) Applicant: Tata Consultancy Services Limited, Mumbai (IN)

(72) Inventors: Sunil NAGPAL, Pune (IN); Mohammed Monzoorul Haque, Pune (IN); Sharmila Shekhar Mande, Pune (IN); Mitali Merchant, Pune (IN); Venkata Siva Kumar Reddy Chennareddy, Hyderabad (IN)

(73) Assignee: Tata Consultancy Services Limited, Mumbai (IN)

(21) Appl. No.: 18/422,276

(22) Filed: Jan. 25, 2024

(30) Foreign Application Priority Data
Apr. 19, 2023 (IN) 202321028609

(51) Int. Cl. C12Q 1/6883; C12Q 1/6851; C12Q 1/689; G16B 40/00

(52) U.S. Cl. CPC C12Q 1/6883; C12Q 1/6851; C12Q 1/689; G16B 40/00; C12Q 2600/106; C12Q 2600/118

(57) ABSTRACT

This disclosure relates more particularly to risk assessment of autism spectrum disorder (ASD) present in the subject and designing a personalized recommendation for the same. Current diagnostic tools and procedures, though abundant in numbers, are all based on psychiatric or behavioral evaluations, checklists and associated statistical inferences, which highlight the inherent limitation in making a reliable and early diagnosis. The present disclosure makes use of oral microbial samples of both saliva and dental plaque. The present disclosure involves a paired extraction and quantification of site-specific unique microbial sequences pertaining to the oral microbial samples of an ASD subject and subsequent classification of the subject under the ASD risk category using a metric based on a predefined ensemble of mathematical formulas. Further, a guided development of personalized microbial cocktail(s) is then designed based on the most relevant formula-set for the subject.

### PRIORITY CLAIM

This U.S. patent application claims priority under 35 U.S.C. § 119 to: Indian Patent Application No. 202321028609, filed on Apr. 19, 2023. The entire contents of the aforementioned application are incorporated herein by reference.

### TECHNICAL FIELD

The disclosure herein generally relates to a risk assessment of disorders present in a subject and, more particularly, to method and system for a risk assessment of autism spectrum disorder (ASD) present in the subject and designing a personalized recommendation for the same.

### SEQUENCE LISTING

This application contains a Sequence Listing which has been submitted in ST.26 format via EFS-Web and is hereby incorporated by reference in its entirety. The ST.26 copy, created on Jan. 12, 2024, is named Sequence_listingRisk assessment for autism spectrum disorder_US.xml and is 21,976 bytes in size.

### BACKGROUND

Autism spectrum disorder (ASD) refers to a spectrum of serious neurodevelopmental or pervasive developmental disorders especially in children that impair their social, communicative, linguistic, cognitive, and behavioral traits. With a worldwide (mean) prevalence of 1 in every 160 children (WHO, 2017), and more than 3.5 million identified cases in US alone, methods for assessing and treating this disorder has long eluded the scientific community. The ASD affected children seek and require significantly higher care and expenses, which include regular monitoring of the condition, routine therapy, special education, and associated healthcare expenses. As per a report on the economic costs of ASD, US spent a total of approximately $11.5 billion in 2011.

Currently available ASD related diagnostic tools and procedures, though abundant in numbers, are predominantly based on psychiatric or behavioral evaluations, checklists and associated statistical inferences, which highlight the inherent limitation in making a reliable and early diagnosis. Several therapeutic regimens have also been attempted in the last few decades, including from use of psychedelic drugs like LSD (1960s), electric shock treatments (in 1970s) to the administration of FDA approved antipsychotic medicines like risperidone, which have focused more (rather completely) on temporary amelioration of ASD related behavior(s) than the cure. Although recent literature exists pertaining to oral microbiome as a possible avenue towards diagnosis and treatment of ASD, it mainly represents a subjective approach and is primarily aimed at comparative analysis of controls and subjects having ASD. Hence, the conventional techniques and procedures for risk assessment and treatment of ASD are not efficient and temporary in nature.

### SUMMARY

Embodiments of the present disclosure present technological improvements as solutions to one or more of the above-mentioned technical problems recognized by the inventors in conventional systems.

In an aspect, a method for risk assessment of autism spectrum disorder in a subject is provided. The method comprising the steps of: collecting a saliva sample and a dental plaque sample of the subject whose risk of autism spectrum disorder is to be assessed; extracting microbial deoxyribonucleic acid (DNA) sequences from each of the saliva sample and the dental plaque sample, individually; determining a quantitative abundance of: (i) each of a plurality of predetermined microbes associated with the saliva sample and (ii) each of a plurality of predetermined microbes associated with the dental plaque sample, individually, from respective extracted DNA sequences, using a first set of probes and a second set of probes specific to each of the plurality of predetermined microbes associated with the saliva sample and the dental plaque sample respectively, through a multiplexed quantitative Polymerase Chain Reaction (qPCR) technique; collating the quantitative abundance of: (i) each of the plurality of predetermined microbes associated with the saliva sample and (ii) each of the plurality of predetermined microbes associated with the dental plaque sample, to obtain a hybrid abundance matrix; determining a model score based on the hybrid abundance matrix, using a pre-determined machine learning (ML) model; performing risk assessment of the subject, based on the model score and a predefined threshold value; and designing, a personalized recommendation for the subject assessed as having autism spectrum disorder, by utilizing a set of rules for the set of microbes that constitute the pre-determined machine learning model to identify one or more personalized probiotic and antibiotic candidates that ameliorate disease symptoms in the subject identified as having autism spectrum disorder.

In yet another aspect, a kit for risk assessment of autism spectrum disorder in a subject is provided. The kit comprising: an input module for receiving a saliva sample and a dental plaque sample of the subject whose risk of autism spectrum disorder is to be assessed; one or more hardware processors configured to analyze the saliva sample and the dental plaque sample; and an output module for displaying the risk assessment of autism spectrum disorder of the subject, based on the analysis of the one or more hardware processors.

In an embodiment, the plurality of predetermined microbes associated with the saliva sample comprises of Mogibacterium, Peptostreptococcus, Eubacterium, Solobacterium, Actinomyces, and Alistipes; and the plurality of predetermined microbes associated with the dental plaque sample comprises of Eubacterium, Dialister, Atopobium, Enterococcus, Mogibacterium, and Anaeroglobus.

In an embodiment, the first set of probes specific to each of the plurality of predetermined microbes associated with the saliva sample are utilized in a first multiplexed qPCR run, and a second multiplexed qPCR run, to determine the quantitative abundance of each of the plurality of predetermined microbes associated with the saliva sample, and wherein:
(i) the plurality of predetermined microbes, the quantitative abundance of which are being determined through the first multiplexed qPCR run are: Mogibacterium, Peptostreptococcus, Eubacterium, and Solobacterium; and
(ii) the plurality of predetermined microbes, the quantitative abundance of which are being determined through the second multiplexed qPCR run are: Mogibacterium, Peptostreptococcus, Actinomyces, and Alistipes.

In an embodiment, the second set of probes specific to each of the plurality of predetermined microbes associated with the dental plaque sample are utilized in a third multiplexed qPCR run, and a fourth multiplexed qPCR run, to determine the quantitative abundance of each of the plurality of predetermined microbes associated with the dental plaque sample, and wherein:
(i) the plurality of predetermined microbes, the quantitative abundance of which are being determined through the third multiplexed qPCR run are: Eubacterium, Dialister, Atopobium, and Enterococcus; and
(ii) the plurality of predetermined microbes, the quantitative abundance of which are being determined through the fourth multiplexed qPCR run are: Eubacterium, Dialister, Mogibacterium, and Anaeroglobus.

In an embodiment, the pre-determined machine learning (ML) model is an ensemble ML model that is built using a microbial abundance data corresponding to a plurality of training saliva samples and a plurality of training dental plaque samples.

In an embodiment, the plurality of predetermined microbes associated with the saliva sample and the plurality of predetermined microbes associated with the dental plaque sample are features of the pre-determined machine learning (ML) model.

In an embodiment, one or more predetermined microbes out of the plurality of predetermined microbes associated with the saliva sample, are common to the first multiplexed qPCR run and the second multiplexed qPCR run for determining the quantitative abundance, and wherein the one or more predetermined microbes that are common to the first multiplexed qPCR run and the second multiplexed qPCR run are determined based on (i) a median abundance of each of the plurality of predetermined microbes obtained from the plurality of training saliva samples, (ii) a frequency of occurrence of each of the plurality of predetermined microbes constituting the ensemble ML model.

In an embodiment, one or more predetermined microbes out of the plurality of predetermined microbes associated with the dental plaque sample are common to the third multiplexed qPCR run and the fourth multiplexed qPCR run for determining the quantitative abundance, and wherein the one or more predetermined microbes that are common to the third multiplexed qPCR run and the fourth multiplexed qPCR run are determined based on (i) a median abundance of each of the plurality of predetermined microbes obtained from the plurality of training dental plaque samples, (ii) a frequency of occurrence of each of the plurality of predetermined microbes constituting the ensemble ML model.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates an exemplary block diagram of a system for risk assessment of autism spectrum disorder present in the subject, according to some embodiments of the present disclosure.

FIGS. 2A and 2B are flowcharts illustrating a method for risk assessment of autism spectrum disorder present in the subject, according to some embodiments of the present disclosure.

FIG. 3A illustrates an exemplary probe and multiplexed qPCR design for detecting and determining the quantitative abundance of each of a plurality of predetermined microbes associated with a saliva sample, according to some embodiments of the present disclosure.

FIG. 3B illustrates an exemplary probe and multiplexed qPCR design for detecting and determining the “quantitative abundance of each of the plurality of predetermined microbes associated with a dental plaque sample, according to some embodiments of the present disclosure.

FIGS. 4A, 4B and 4C are flowcharts illustrating steps involved in building a pre-determined machine learning model according to some embodiments of the present disclosure.

FIG. 5 illustrates an exemplary block diagram of a kit for risk assessment of autism spectrum disorder present in the subject, according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments.

Role of brain's cerebral cortex in deciding the intelligence, behavior, personality, perception, and language processing capabilities of an individual is very well established. Several studies have rather attempted to understand the changes in cerebral cortex of autism spectrum disorder (ASD) affected subjects as well. Given the fact that oral tissue has dense sensory innervations connecting the oral environment to the cerebral cortex, the hypothesis that a change in oral microbiota and metabolites thereof could have profound impact on functioning of cerebral cortex, begs due attention.

Last 15 years have also seen a huge surge in interest towards understanding the role of human microbiome in various disease states. Gut microbiome in particular has received a lot of attention and innumerable studies have established firm scientific evidence linking the changes in gut microbiota with onset, progression and/or even reversal of disease states. The theory of gut-brain axis, focusing on the duplex communication between the central and the enteric nervous system, and a consequent connection of emotional and cognitive centers of brain with the functioning of intestine(s) particularly got an intriguing turn when the idea of the effects of gut microbiota on intestinal function (and hence the nervous function) is disclosed. Ever since, multitude of scientific groups have explored this paradigm of the effects of gut microbes on brain behavior, especially in the context of neurodevelopmental and neurodegenerative disorders. Alterations in the gut microbial community structure of ASD subjects have consistently been reported in the recent past. There are conventional techniques emerged that make use of specific gut microbial species as prospective therapeutic or diagnostic markers for ASD. A reliable translatable product is yet to reach the market though.

Some of the existing diagnostic and therapeutic methods, and their limitations are summarized below:
(i) Psychiatric or behavioral evaluations, checklists and associated statistical inferences, even though constitute the prevailing and accepted state of art in diagnosis, they merely represent qualitative diagnosis based on human judgments. No physical trait or behavior can be the sole and unique representative of the actual physiological state of mind. Even the diagnostic evaluations and checklists themselves make a declaration that the outcomes of the tests are not conclusive evidence of the ASD.
(ii) Diagnostic tests for the ASD are not available or very limited to the above-mentioned evaluative checklists.
(iii) Microbiome as a biomarker for the ASD has been attempted, but most of them are confined to gut microbiome with limited accuracies.
(iv) Oral microbiome as a biomarker for the ASD is present, but mainly represents a subjective approach aimed at comparative analysis of controls and ASD subjects. A random forest-based approach for classification of samples relies on as high as 36 Operational Taxonomic Units (OTUs) or microbes in saliva samples alone and as high as 54 OTUs or microbes in plaque samples alone. Dependence on a large number of microbial features for developing a diagnostic test is neither easily translatable nor economical. Moreover, it is difficult to arrive at a relevant and relatable therapeutic solution based on such a large number of microbes in a diagnostic marker.
(v) Several therapeutic regimes have been attempted in the last (more than) 100 years of ASD's history, ranging from use of psychedelic drugs like LSD (1960s), electric shock treatments (in 1970s) to the administration of FDA approved anti-psychotic medicines like risperidone, which have focused more (rather completely) on temporary amelioration of behavior(s) than the cure. In the current state of art, no therapeutic drug or supplement exists for curing the ASD.

The present disclosure solves the challenges present in the existing state of art, for accurate identification and selection of subjects with increased risk of the ASD and providing a personalized microbial cocktail for treating the ASD, using oral microbiome of the ASD affected subjects. The present disclosure attempts to offer an early, easy and reliable risk assessment and a method for design of personalized recommendable composition for applications towards amelioration of ASD.

The present disclosure involves a paired extraction and quantification of site-specific unique microbial sequences (pertaining to the oral microbial samples) of an ASD subject and subsequent classification of the subject under the ASD risk category using a metric based on a predefined ensemble of mathematical formulas (using ensemble machine learning approach) specific to some unique (not all) combinations of extracted microbial sequences. Each formula in the entire ensemble contains a mix of microbial sequences from the microbial sample sites. Further, a guided development of personalized recommended microbial cocktail (s) is then carried out based on the most relevant formula-set for the subject. The identification of the formula ensemble, its constituting combination of microbial sequences, and associated mathematical relationships and constants/coefficients, is dependent on an independently devised ensemble machine learning approach.

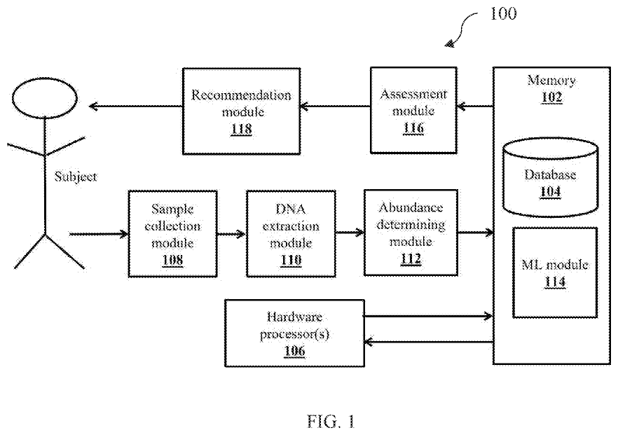

Referring now to the drawings, and more particularly to FIG. 1 through 5, where similar reference characters denote corresponding features consistently throughout the figures, there are shown preferred embodiments and these embodiments are described in the context of the following exemplary system and/or method.

FIG. 1 illustrates an exemplary block diagram of a system 100 for risk assessment of autism spectrum disorder present in the subject, according to some embodiments of the present disclosure. In an embodiment, the system 100 includes a memory 102, a database 104, one or more hardware processors 106, a sample collection module 108, a DNA extraction module 110, an abundance determining module 112, a machine learning (ML) module 114, an assessment module 116, and a recommendation module 118. In an embodiment, the database 104 and the machine learning (ML) module 114 are stored in the memory 102.

In an embodiment, the sample collection module 108 is configured to collect a saliva sample and a dental plaque sample of the subject whose risk of autism spectrum disorder is to be assessed. The DNA extraction module 110 is configured to extract microbial deoxyribonucleic acid (DNA) sequences from each of the saliva sample and the dental plaque sample, individually. The abundance determining module 112 is configured to determine a quantitative abundance of: (i) each of a plurality of predetermined microbes associated with the saliva sample and (ii) each of a plurality of predetermined microbes associated with the dental plaque sample, individually, from respective extracted DNA sequences.

The machine learning (ML) module 114 is configured to determine a model score based on the hybrid abundance matrix which is obtained by collating the quantitative abundance of: (i) each of the plurality of predetermined microbes associated with the saliva sample and (ii) each of the plurality of predetermined microbes associated with the dental plaque sample. The assessment module 116 is configured to perform risk assessment of the subject, based on the model score. Lastly, the recommendation module 118 is configured to design a personalized recommendation for the subject assessed as having the autism spectrum disorder.

In an embodiment, the one or more hardware processors 106 can be implemented as one or more microprocessors, microcomputers, microcontrollers, digital signal processors, central processing units, state machines, logic circuitries, and/or any devices that manipulate signals based on operational instructions. Among other capabilities, the one or more hardware processors 106 is configured to fetch and execute computer-readable instructions stored in the memory 102. In an embodiment, the system 100 can be implemented in a variety of computing systems including laptop computers, notebooks, hand-held devices such as mobile phones, workstations, mainframe computers, servers, a network cloud and the like.

The memory 102 may include any computer-readable medium known in the art including, for example, volatile memory, such as static random-access memory (SRAM) and dynamic random-access memory (DRAM), and/or non-volatile memory, such as read only memory (ROM), erasable programmable ROM, flash memories, hard disks, optical disks, and magnetic tapes.

Further, the memory 102 may include a database 104 configured to include information regarding risk assessment of autism spectrum disorder present in the subject. The memory 102 may comprise information pertaining to input(s)/output(s) of each step performed by the one or more hardware processors 106 of the system 100 and methods of the present disclosure. In an embodiment, the database 104 may be external (not shown) to the system 100 and coupled to the system 100 via the I/O interfaces (not shown in FIG. 1).

In an embodiment, one or more data storage devices or the memory 102 operatively coupled to the one or more hardware processors 106. The system 100 with the one or more hardware processors 106 is configured to execute functions of one or more functional modules of the system 100.

The system 100 supports various connectivity options such as BLUETOOTH®, USB, ZigBee and other cellular services. The network environment enables connection of various components of the system 100 using any communication link including Internet, WAN, MAN, and so on. In an exemplary embodiment, the system 100 is implemented to operate as a stand-alone device. In another embodiment, the system 100 may be implemented to work as a loosely coupled device to a smart computing environment. The components and functionalities of the system 100 are described further in detail.

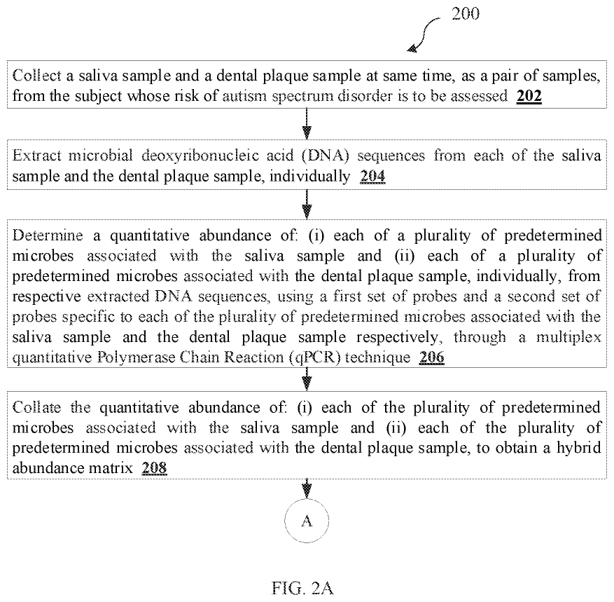

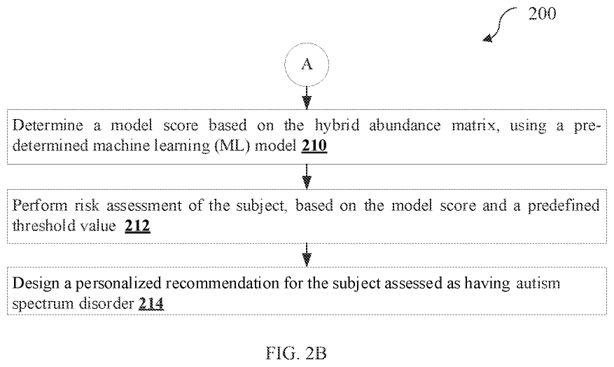

In an embodiment, the memory 102 comprises one or more data storage devices operatively coupled to the one or more hardware processors 106 and is configured to store instructions for execution of steps of the method depicted in FIGS. 2A and 2B by the one or more hardware processors 106. FIGS. 2A and 2B are flowcharts illustrating a method 200 for risk assessment of autism spectrum disorder present in the subject, according to some embodiments of the present disclosure. The steps of the method 200 of the present disclosure will now be explained with reference to the components or blocks of the system 100 as depicted in FIG. 1 and the steps of flow diagrams as depicted in FIGS. 2A and 2B. Although process steps, method steps, techniques or the like may be described in a sequential order, such processes, methods, and techniques may be configured to work in alternate orders. In other words, any sequence or order of steps that may be described does not necessarily indicate a requirement that the steps to be performed in that order. The steps of processes described herein may be performed in any order practical. Further, some steps may be performed simultaneously.

At step 202 of the method 200, the saliva sample and the dental plaque sample of the subject whose risk of the autism spectrum disorder (ASD) is to be assessed, are collected through the sample collection module 108. In an embodiment, the subject or the individual is a human being. Both the saliva sample and the dental plaque sample are collected (together) as a pair at the same time, from the same subject whose risk of the ASD is to be assessed. As the ASD is very important to be assessed especially in children, the subject preferably is a human child. However, the adoption of similar sampling, assessment and recommendation strategy in other age groups is well within the scope of the present disclosure.

Both the saliva sample and the dental plaque sample are oral microbial and site-specific samples and are non-invasive. In an embodiment, the saliva sample may refer to extracted salivary swabs or naturally out-flown saliva or voluntarily spitted saliva which is obtained in a non-stimulatory environment (where stimulations refer to behavioral or digestive triggers). In general, the saliva sample is extracted from the mouth site of the subject. The dental plaque samples refer to the scraps obtained from oral site-specific area of the subject where the teeth are present. In an embodiment, the scraps can be obtained using instruments such as curettes.

Further, at step 204 of the method 200, microbial deoxyribonucleic acid (DNA) sequences from each of the saliva sample and the dental plaque sample collected at step 202 of the method 200, are extracted individually through the DNA extraction module 110. In an embodiment, the extraction of microbial DNA sequences from both the saliva sample and the dental plaque sample is performed by amplification of 16S rRNA marker genes (either full-length or specific variable regions of the gene) using one or more of: a next-generation sequencing (NGS) platform, Oxford nanopore sequencing or any other DNA sequencing technique and platform (including a classical Sanger sequencing). In another embodiment, the NGS platforms include any one of whole genome sequencing, CPN60 gene-based amplicon sequencing, other phylogenetically conserved genetic region-based amplicon sequencing, sequencing using approaches which involve either a fragment library or a mate-pair library or a paired-end library or a combination of the same. Further, the DNA extraction module 110 includes taxonomic classification of the sequenced reads at genus level using RDP, and latest version of any other taxonomic classification database such as Greengenes or Silva databases, or algorithms such as dada2 are covered in the scope of this invention

Further, at step 206 of the method 200, the quantitative abundance of: (i) each of a plurality of predetermined microbes associated with the saliva sample and (ii) each of a plurality of predetermined microbes associated with the dental plaque sample, is determined individually, from respective extracted DNA sequences extracted at step 204 of the method 200. More specifically, the quantitative abundance of each of the plurality of predetermined microbes associated with the saliva sample is determined from the microbial DNA sequences corresponding to the saliva sample. Similarly, the quantitative abundance of each of the plurality of predetermined microbes associated with the dental plaque sample is determined from the microbial DNA sequences corresponding to the dental plaque sample. The plurality of predetermined microbes is the corresponding operational taxonomy units (OTUs) whose quantification is performed in the corresponding sample.

In an embodiment, the plurality of predetermined microbes associated with the saliva sample are: Mogibacterium, Peptostreptococcus, Eubacterium, Solobacterium, Actinomyces, and Alistipes. In an embodiment, the plurality of predetermined microbes associated with the dental plaque sample are: Eubacterium, Dialister, Atopobium, Enterococcus, Mogibacterium, and Anaeroglobus. The scope of the present disclosure is not limited to the mentioned names of the predetermined microbes and applicable to other alternative names of the microbes present in the art.

The marker feature sequences of some of the predetermined microbes are listed below:

### Mogibacterium:

| | |
|---|---|
| 1 | gatgaacgct ggcggcgtgc ctaatacatg caagtcgagc gagaagcctg gaaatgacgc |
| 61 | ttcggttgaa tttccaagcg gacagcggcg gacgggtgag taacgcgtag gcaacctgcc |
| 121 | cctgtacaga gggatagcca ttggaaacga tgattaaaac ctcatgacac cgtagtagca |
| 181 | catgctacat cggtcaaaga tttatcggtc agggatgggc ctccgtctga ttaactggtt |
| 241 | ggtgaggtaa cggctcacca aggtgacgat cagtagccga cctgagaggg tgatcggcca |
| 301 | cattggaact gagacacggt ccaaacttct acggaaggca gcagtaggga atcttgcaca |
| 361 | atgggcgtaa gcctgatgca gcaacgccgc gtgaaggatg aaggcttcgg gttgtaaact |
| 421 | tctgttctaa gggaagaaag aaatgacggt accttaggag caagccccgg ctaactacgt |
| 481 | gccagcagcc gcggtaatac gtagggggca agcgttatcc ggaattattg ggcgtaaaga |
| 541 | gtgcgtaggt ggttacctaa gcgcaaggtt taaattagag gctcaacctc tacatgcctt |
| 601 | gcgaactggg ctacttgagt gcaggagggg aaagcggaat tcctagtgta gcggtgaaat |
| 661 | gcgtagatat taggaggaac accggcggcg aaggcggctt tctggactgt aactgacact |
| 721 | gaggcacgaa agcgtgggta gcaaacagga ttagataccc tggtagtcca cgccgtaaac |
| 781 | gatgagcact aggtgttggg tccgttagga ctcagtgccg cagttaacgc aataagtgtc |
| 841 | cgcctgggag tacgctcgca agagtaaact caaggaattg acgggcaccc gcacaagcag |
| 901 | gggagcttgt ggtttaattc gaagcaacgc gaagaacctt accagggctt gacatcctgc |
| 961 | tgacagaacc ttaatcggtt ttttcttcgg acagcagaga caggtggtgc atggttgtcg |
| 1021 | tcagctcgtg tcgtgagatg ttgggttaag tcccgcaacg agcgcaaccc ttgtcgctag |
| 1081 | ttactaacat tcagttgagg actctagcga gactgccgag gtcaactcgg aggaaggtgg |
| 1141 | ggatgacgtc aaatcatcat gccccttatg ttctgggcta cacacgtgct acaatggtcg |
| 1201 | gtacaatgag atgcaatact gcgaagtgga gcgaaacacc aaaaccgatc ccagttcgga |
| 1261 | ttgtaggctg caactcgcct acatgaagtc ggagttgcta gtaatcgcag atcagaatgc |
| 1321 | tgcggtgaat gcgttcccgg gtcttgtaca caccgcccgt cacaccatgg aagttggggg |
| 1381 | tgcccaaagt cggttaatta atctatcgcc taaggcaaaa ccaatgactg gggt |

### Eubacterium:

| | |
|---|---|
| 1 | gtgctgcaga gaagagtttg atcctggctc aggatgaacg |
| | ctggcggcgt gcctaacaca |
| 61 | tgcaagttga gcgagaaatc acttaaagaa gcttcggtag |
| | acttaagaga tggagagcag |
| 121 | cggacgggtg aggaacgcgt gggaaacctg cccttgacag |
| | gaggatagcc gagagaaatt |
| 181 | tcgatttaat acttcataaa gcagagcatt cgcatggatg |
| | aactgccaaa gaattatcgg |
| 241 | tcaaggatgg tccagcgtct gattagctgg ttggtaaggt |
| | accggcttac caaggcgacg |
| 301 | atcagtagcc ggcctgagag ggtgaacggc cacattggaa |
| | ctgagacacg gtccagactc |
| 361 | tacgggaggc agcagtgggg aatattgcac aatgggcgaa |
| | gcctgatgca gcaacgccgc |
| 421 | gtgaaggaag aaggctttcg agtcgtaaac ttctgtccaa |
| | agggaagaat aatgacggta |
| 481 | cctttgaaga aagccccggc taactacgtg ccagcagccg |
| | cggtaatacg tagggggcga |
| 541 | gcgttatccg gaattactgg gcgtaaagag tatgtaggtg |
| | gttaagtaag cgtagggttt |
| 601 | aaggcgacag cccaactgtc gtatgccccg cgagctgttt |
| | aacttgagta caggagggga |
| 661 | aggcggaatt cctagtgtag cgctgaaatg cgtagatatt |
| | aggaggaaca ccagtggcga |
| 721 | aggcggcctt ctggactgta actgacactg agatacgaaa |
| | gcgtgggtag caaacaggat |
| 781 | tagataccct ggtagtccac gccgtaaacg atgagcacta |
| | ggtgtcgggc tcgcaagagt |
| 841 | tcggtgccgg agcaaacgca ttaagtgctc cgcctgggga |
| | gtacgcacgc aagtgtgaaa |
| 901 | ctcaaaggaa ttgacgggga cccgcacaag cagcggagca |
| | tgtggtttaa ttcgaagcaa |
| 961 | cgcgaagaac cttaccagga cttgacatcc cactgaaagc |
| | tcggttaaaa ctgagccctt |
| 1021 | cttcggaaca gtggagacag gtggtgcatg gttgtcgtca |
| | gctcgtgtcg tgagatgttg |
| 1081 | ggttaagtcc cgcaacgagc gcaacccttg ccgttagttg |
| | ccatcattaa gttgggcact |
| 1141 | ctaatgggac tgccggggag aacccggagg aaggcgggga |
| | tgacgtcaaa tcatcatgcc |
| 1201 | ccttatgttc tgggctacac acgtgctaca atggccgtca |
| | cagagggaag cgagagagcg |
| 1261 | atcttaagcg aaaccaaaaa ggcggtccca gttcggactg |
| | caggctgcaa ctcgcctgca |
| 1321 | cgaagccgga gttgctagta atcgcggatc agaatgtcgc |
| | ggtgaatgcg ttcccgggtc |
| 1381 | ttgtacacac cgccggtgga tccgtg |

### Dialister:

| | |
|---|---|
| 1 | cgaacgctgg cggcgtgctt aacacatgca agtcgaacgg aaagagatga aagagcttgc |
| 61 | tcttttatta atttcagtgg caaacgggtg agtaacacgt aaacaacctg ccttaaggat |
| 121 | ggggataaca gacggaaacg actgctaata ccgaatacgt tctaagcatc gcatggtgca |
| 181 | tagaagaaag ggtggcctct acaagaaagc tatcgcctta agaggggttt gcgactgatt |
| 241 | aggtagttgg tgaggtaacg gctcaccaag ccgacgatca gtagccggtc tgagaggatg |
| 301 | aacggccaca ctggaactga gacacggtcc agactcctac gggaggcagc agtggggaat |
| 361 | cttccgcaat ggacgaaagt ctgacggagc aacgccgcgt gaacgaagaa ggtcttcgga |
| 421 | ttgtaaagtt ctgtgattcg ggacgaaagg gtttgtggtg aataatcatt gacattgacg |
| 481 | gtaccgaaaa agcaagccac ggctaactac gtgccagcag ccgcggtaat acgtaggtgg |
| 541 | caagcgttgt ccggaattat tgggcgtaaa gcgcgcgcag gcggtttctt aagtccatct |
| 601 | taaaagcgtg gggctcaacc ccatgagggg atggaaactg ggaagctgga gtatcggaga |
| 661 | ggaaagtgga attcctagtg tagcggtgaa atgcgtagag attaggaaga acaccggtgg |
| 721 | cgaaggcgac tttctagacg aaaactgacg ctgaggcgcg aaagcgtggg gagcaaacag |
| 781 | gattagatac cctggtagtc cacgccgtaa acgatggata ctaggtgtag gaggtatcga |
| 841 | cccctcctgt gccggagtta acgcaataag tatcccgcct gggaagtacg atcgcaagat |
| 901 | taaaactcaa aggaattgac gggggcccgc acaagcggtg gagtatgtgg tttaattcga |
| 961 | cgcaacgcga agaaccttac caagccttga cattgatcgc aatccataga aatatggagt |
| 1021 | tcttcttcgg aagacgagaa aacaggtggt gcacggctgt cgtcagctcg tgtcgtgaga |
| 1081 | tgttgggtta agtcccgcaa cgagcgcaac ccctatttcc tgttaccagc acgtaaaggt |
| 1141 | ggggactcag gagagaccgc cgcggacaac gcggaggaag gcggggatga cgtcaagtca |
| 1201 | tcatgcccct tatggcttgg gctacacacg tactacaatg ggtgccaaca aagagaagcg |
| 1261 | aaatcgcgag atggagcgga cctcataaac gcacccccag ttcagattgc aggctgcaac |
| 1321 | tcgcctgcat gaagtaggaa tcgctagtaa tcgcgggtca gcataccgcg gtgaatacgt |
| 1381 | tcccgggcct tgtacacacc gcccgtcaca ctatgagagt tagagacacc cgaagccggt |
| 1441 | gaggtaaccg caagggacca gccgtctaag gtggagctga tgattggagt gaagtcgtaa |
| 1501 | caag |

### Enterococcus:

| | |
|---|---|
| 1 | agagtttgat cctggctcag gacgaacgct ggcggcgtgc ctaatacatg caagtcgaac |
| 61 | gcttctttcc tcccgagtgc ttgcactcaa ttggaaagag gagtggcgga cgggtgagta |
| 121 | acacgtgggt aacctaccca tcagaggggg ataacacttg gaaacaggtg ctaataccgc |
| 181 | ataacagttt atgccgcatg gcataagagt gaaaggcgct ttcgggtgtc gctgatggat |
| 241 | ggacccgcgg tgcattagct agttggtgag gtaacggctc accaaggcca cgatgcatag |
| 301 | ccgacctgag agggtgatcg gccacactgg gactgagaca cggcccagac tctacgggag |
| 361 | gcagcagtag ggaatcttcg gcaatggacg aaagtctgac cgagcaacgc cgcgtgagtg |
| 421 | aagaaggttt tcggatcgta aaactctgtt gttagagaag aacaaggacg ttagtaactg |
| 481 | aacgtcccct gacggtatct aaccagaaag ccacggctaa ctacgtgcca gcagccgcgg |
| 541 | taatacgtag gtggcaagcg ttgtccggat ttattgggcg taaagcgagc gcaggcggtt |
| 601 | tcttaagtct gatgtgaaag cccccggctc aaccggggag ggtcattgga aactgggaga |
| 661 | cttgagtgca gaagaggaga gtggaattcc atgtgtagcg gtgaaatgcg tagatatatg |
| 721 | gaggaacacc agtggcgaag gcggctctct ggtctgtaac tgacgctgag gctcgaaagc |
| 781 | gtggggagca aacaggatta gataccctgg tagtccacgc cgtaaacgat gagtgctaag |
| 841 | tgttggaggg tttccgccct tcagtgctgc agcaaacgca ttaagcactc cgcctgggga |
| 901 | gtacgaccgc aaggttgaaa ctcaaaggaa ttgacggggg cccgacaagc ggtggagcat |
| 961 | gtgtttaatt cgaagcaacg cgaagaacct taccaggtct tgacatcctt tgaccactct |
| 1021 | agagatagag ctttcccttc ggggacaaag tgacagtggt gcatggttgt cgtcagctcg |
| 1081 | tgtcgtgaga tgttgggtta agtccgcaac gagcgcaacc cttattgtta gttgccatca |
| 1141 | tttagttggg cactctagcg agactgccgg tgacaaaccg gaggaaggtg gggatgacgt |
| 1201 | caaatcatca tgccccttat gacctgggct acacacgtgc tacaatggga agtacaacga |
| 1261 | gtcgctagac cgcgaggtca tgcaaatctc ttaaagcttc tctcagttcg gattgcaggc |
| 1321 | tgcaactcgc ctgcatgaag ccggaatcgc tagtaatcgc ggatcagcac gccgcggtga |
| 1381 | atacgttccc gggccttgta cacaccgccc gtcacaccac gagagtttgt aacacccgaa |
| 1441 | gtcggtgagg taaccttttt ggagccagcc gcctaaggtg ggatagatga tttgggtgaa |
| 1501 | gtcgtaacaa ggtaacc |

### Anaeroglobus:

| | |
|---|---|
| 1 | cttacgatcg agtggcaaac gggtgagtaa cgcgtaaaca acctgccccg cagatgggga |
| 61 | caacagctgg aaacggctgc taataccgaa tacggtcctc ttagcgcatg gtaagaggaa |
| 121 | gaaagggtgg cctctggaac aagctaccgc tgtgggaggg gtttgcgtct gattagctgg |
| 181 | ttggaggggt aacggcccac caaggcgacg atcagtagcc ggtctgagag gatgaacggc |
| 241 | cacattggaa ctgagacacg gtccagactc ctacgggagg cagcagtggg gaatcttccg |
| 301 | caatgggcga aagcctgacg gagcaacgcc gcgtgagtga agacggcctt cgggttgtaa |
| 361 | agctctgtta taggggacga acggccgggt agcgaagagg tagccggcat gacggtaccg |
| 421 | taagagaaag ccacggctaa ctacgtgcca gcagccgcgg taatacgtag gtggcaagcg |
| 481 | ttgtccggaa tgattgggcg taaagggcgc gcaggcggct gtgtaagtct gtctaaaaag |
| 541 | tgcggggcta aaccccgtga gaggatggaa actggacagc tgagagtgtc ggagaggaaa |
| 601 | gcggaattcc tagtgtagcg gtgaaatgcg tagatattag gaggaacacc ggtggcgaaa |
| 661 | gcggctttct ggacgacaac tgacgctgag gcgcgaaagc caggggagca aacgggatta |
| 721 | gataccccgg tagtcctggc cgtaaacgat gggtactagg tgtaggaggt atcgacccct |
| 781 | cctgtgccgg agttaacgca ataagtaccc cgcctgggga gtacggccgc aaggctgaaa |
| 841 | ctcaaaggaa ttgacggggg cccgcacaag cggtggagta tgtggtttaa ttcgacgcaa |
| 901 | cgcgaagaac cttaccaagc cttgacattg ctcgcaacgg gtagagatac ctggttcttc |
| 961 | ttcggaagac gagacaacag gtggtgcacg gctgtcgtca gctcgtgtcg tgagatgttg |
| 1021 | ggttaagtcc cgcaacgagc gcaaccccta tcttcagtta ccagcacgta gcggtgggga |
| 1081 | ctcaggagag acagccgcag acaatgcgga ggaaggcggg gacgacgtca agtcatcatg |
| 1141 | ccccttatgg cttgggctac acacgtacta caatggctct aaatagaggg aagcgaagga |
| 1201 | gcgatccgga gcaaaccccg caaacagagt cccagttcgg attgcaggct gcaactcgcc |
| 1261 | tgcatgaagg aggaatcgct agtaatcgca ggtcagcata ctgcggtgaa tacgttcccg |
| 1321 | ggccttgtac acaccgcccg tcacaccacg aaagtcattc acacccgaag ccggtgaggt |
| 1381 | aaccgcaagg agccagccgt caaaggtg |

### Peptostreptococcus:

| | |
|---|---|
| 1 | gatgaacgct ggcggcgtgc ctaacacatg caagtcgagc gagggtttgc tcagtattga |
| 61 | gtattctaag actagaatgt tcaattctga gcaaaaccaa gcggcggacg ggtgagtaac |
| 121 | gcgtgggtaa cctgccctat acacatggat aacatactga aaagtttact aatacatgat |
| 181 | aatatatatt tgcggcatcg cagatatatc aaagtgttag cggtatagga tggacccgcg |
| 241 | tctgattagc tagttggtga gataactgcc caccaaggcg acgatcagta gccgacctga |
| 301 | gagggtgatc ggccacattg gaactgagac acggtccaaa ctcctacggg aggcagcagt |
| 361 | ggggaatatt gcacaatggg cgaaagcctg atgcagcaac gccgcgtgaa cgatgaaggt |
| 421 | cttcggatcg taaagttctg ttgcagggga agataatgac ggtaccctgt gaggaagccc |
| 481 | cggctaacta cgtgccagca gccgcggtaa tacgtagggg gctagcgtta tccggattta |
| 541 | ctgggcgtaa agggtgcgta ggtggtcctt caagtcggtg gttaaaggct acggctcaac |
| 601 | cgtagtaagc cgccgaaact ggaggacttg agtgcaggag aggaaagtgg aattcccagt |
| 661 | gtagcggtga aatgcgtaga tattgggagg aacaccagta gcgaaggcgg ctttctggac |
| 721 | tgcaactgac actgaggcac gaaagcgtgg gtagcaaaca ggattagata ccctggtagt |
| 781 | ccacgctgta aacgatgagt actaggtgtc gggggttacc cccctcggtg ccgcagctaa |
| 841 | cgcattaagt actccgcctg gggagtacgc acgcaagtgt gaaactcaaa ggaattgacg |
| 901 | gggacccgca caagtagcgg agcatgtggt ttaattcgaa gcaacgcgaa gaaccttacc |
| 961 | taagcttgac atccctcgga caggtgttta atcacaccct tccttcggga ctgaggtgac |
| 1021 | aggtggtgca tggttgtcgt cagctcgtgt cgtgagatgt tgggttaagt cccgcaacga |
| 1081 | gcgcaaccct tgtctttagt tgccagcatt cagttgggca ctctagagag actgccaggg |
| 1141 | ataacctgga ggaaggtggg gatgacgtca aatcatcatg ccccttatgc ttagggctac |
| 1201 | acacgtgcta caatgggtgg tacagagggt tgccaaaccg tgaggtggag ccaatccctt |
| 1261 | aaagccattc tcagttcgga ttgtaggctg aaactcgcct acatgaagct ggagttacta |
| 1321 | gtaatcgcag atcagaatgc tgcggtgaat gcgttcccgg gtcttgtaca caccgcccgt |
| 1381 | cacaccacgg gagtcggaaa cacccgaagc cgattatcca accgcaagga ggaagtcgtc |
| 1441 | gaaggtggcg tcnataactg gggtg |

### Solobacterium:

| | |
|---|---|
| 1 | agagtttgat cctggctcag tatgaactct gccggcgtgc ctaatacttg caagtcgaac |
| 61 | gctgaagatc tagcttgcta gatcgaagga gtgccgaacg ggtgagtaat acataagcaa |
| 121 | cctacccacg aagactggga taatctctgg aaacggggac taataccgga taggtaatcg |
| 181 | gaaggcatct tctggttatt aaaggttaaa aacactggtg gatgggctta tggcgcatta |
| 241 | gttagttggt gaggtaacgg cccaccaaga cgatgatgcg tagccggcct gagagggtga |
| 301 | acggccacat tgggactgag acacggccca gactctacgg gaggcagcag tagggaattt |
| 361 | tcggcaatag gggcaaccct gaccgagcaa cgccgcgtga gtgaagacgg ccttcgggtt |
| 421 | gtaaaagctc ttgtttgtaa gggaagaacg gtagatagag aatatctaag tgacggtacc |
| 481 | ttaccagaaa gccacggcta actacgtgcc agcagccgcg gtaatacgta ggtggcgacc |
| 541 | gttatccgga attattggcc gtaaagggtg cgtaggcggc ctgttaagta agtggttaaa |
| 601 | ttgttgggct caacccaatc cagccactta aactggcagg ctagagtatt ggagaggcaa |
| 661 | gtggaattcc atgtgtagcg gtaaaatgcg tagatatatg gaggaacacc agtggcgaag |
| 721 | gcggcttgct agccaaagac tgacgctcat gcacgaaacc gtggggagcc aataggatta |
| 781 | gataccctag tagtccacgc cgtaaccgat gaatactaag tattggggaa actcagtgct |
| 841 | gcactaacgc aataagtatt ccgcctgtgg agtatgcacg caagtgtgaa acataaagga |
| 901 | attgacgggg gcccgcacaa gcggtggagt atgtggttta attcgacgca acgcgaagaa |
| 961 | ccttaccagg ccttgacatc ccttgcaaag ctgtagagat acagtagagg ttatcaagga |
| 1021 | gacaggtggt tgcatggtgt cgtcagctcg tgtcgtgaga tgttgggtta agtccggcaa |
| 1081 | cgagcgcaac ccttgccttc agttaccagc atttagttgg ggactctgga gggactgccg |
| 1141 | gtgataaacc ggaggaaggt ggggatgacg tcaaatcatc atgcccctta tggcctgggc |
| 1201 | tacacacgta ctacaatggc tgttacaacg tgcagcgacc tagcgatagg aagcgaatca |
| 1261 | ctaaaagaca gtctcagttc ggattgaagt ctgcaactcg acttcatgaa gctggaatcg |
| 1321 | ctagtaactg cggatcagaa tgccgcggtg aatacgttct cgggccttgt acacaccgcc |
| 1381 | cgtcatacca tgagagctgg taatacccga ggccggtggc ctaaccgcaa ggaaggagcc |
| 1441 | gtcgaaggta ggactagtga ttggggtcaa gtcgtaacaa ggtaacc |

### Actinomyces:

| | |
|---|---|
| 1 | tcctgactca ggacgaacgc tgccggcgta cataacacat gcaagtcgaa cggtgaaggg |
| 61 | gcctgctttt gtgggtcctg gatgagtggc gaacgggtga gtaacacgtg agtaacctgc |
| 121 | ccccttcttc tggataaccg catgaaagtg tggctaatac gggatattct gggtctgtcg |
| 181 | catggtgggc ctgggaaaga ttgcgccttt tttggtgttt ttggtggggg atgggctcgc |
| 241 | ggcctatcag cttgttggtg gggtgatggc ctgccaaggc ggtgacgggt agccggcctg |
| 301 | agagggtgga cggtcacact gggactgaga cacggcccag actcctacgg gaggcagcag |
| 361 | tggggaatat tgcacaatgg gcgcaagcct gatgcagcga cgccgcgtga gggatggagg |
| 421 | ccttcgggtt gtgaacctct ttcgccagtg aagcaggcct gcctcgtttg tgggtgggtt |
| 481 | gacggtagct ggataagaag cgccggctaa ctacgtgcca gcagccgcgg taatacgtag |
| 541 | ggcgcgagcg ttgtccggaa ttattgggcg taaagagctc gtaggcggct ggtcgcgtct |
| 601 | gtcgtgaaat cctctggctt aactgggggc ttgcggtggg tacgggccgg cttgagtgcg |
| 661 | gtaggggaga ctggaactcc tggtgtagcg gtggaatgcg cagatatcag gaagaacacc |
| 721 | ggtggcgaag gcgggtctct gggccgttac tgacgctgag gagcgaaagc gtggggagcg |
| 781 | aacaggatta gataccctgg tagtccacgc cgtaaacgtt gggcactagg tgtggggggc |
| 841 | cttttccggg tcttccgcgc cgtagctaac gcattaagtg ccccgcctgg ggagtacggc |
| 901 | cgcaaggcta aaactcaaag gaattgacgg gggcccgcac aagcggcgga gcatgcggat |
| 961 | taattcgatg caacgcgaag aaccttacca aggcttgaca tgtgccggtc tgctccggag |
| 1021 | acggggtttc ctcctttgtg ggggctggtt cacaggtggt gcatggttgt cgtcagctcg |
| 1081 | tgtcgtgaga tgttgggtta agtcccgcaa cgagcgcaac ccttgtctcg tgttgccagc |
| 1141 | acgttgtggt ggggactcgc gggagactgc cggggtcaac tcggaggaag gtggggatga |
| 1201 | cgtcaaatca tcatgcccct tatgtcttgg gcttcacgca tgctacaatg gccggtacag |
| 1261 | agggctgcga taccgtgagg tggagcgaat cccttaaagc cggtctcagt tcggatcggt |
| 1321 | gtctgcaact cgacaccgtg aagttggagt cgctagtaat cgcagatcag caacgctgcg |
| 1381 | gtgaatacgt tctcgggcct tgtacacacc gcccgtcacg tcatgaaagt cggcaacacc |
| 1441 | cgaagcccgt |

### Alistipes:

| | |
|---|---|
| 1 | gatgaacgct agcggcaggc ttaacacatg caagtcgagg ggcagcacga ggtagcaata |
| 61 | ctttggtggc gaccggcgca cgggtgcgta acgcgtatgc aacctacctt taacaggggc |
| 121 | ataacactga gaaattggta ctaattcccc ataacattcg agaaggcatc ttcttgggtt |
| 181 | aaaaactccg gtggttaaag atgggcatgc gttgtattag ctagttggtg aggtaacggc |
| 241 | tcaccaaggc aacgatacat agggggactg agaggttaac cccccacatt ggtactgaga |
| 301 | cacggaccaa actcctacgg gaggcagcag tgaggaatat tggtcaatgg acgcaagtct |
| 361 | gaaccagcca tgccgcgtgc aggaagacgg ctctatgagt tgtaaactgc ttttgtacta |
| 421 | gggtaaacgc tcttacgtgt aggagcctga aagtatagta cgaataagga tcggctaact |
| 481 | ccgtgccagc agccgcggta atacggagga tccaagcgtt atccggattt attgggttta |
| 541 | aagggtgcgt aggcggtttg ataagttaga ggtgaaatac cggggctcaa ctccggaact |
| 601 | gcctctaata ctgttgaact agagagtagt tgcggtaggc ggaatgtatg gtgtagcggt |
| 661 | gaaatgctta gagatcatac agaacaccga ttgcgaaggc agcttaccaa actatatctg |
| 721 | acgttgaggc acgaaagcgt ggggagcaaa caggattaga taccctggta gtccacgcag |
| 781 | taaacgatga taactcgttg tcggcgatac acagtcggtg actaagcgaa agcgataagt |
| 841 | tatccacctg gggagtacgt tcgcaagaat gaaactcaaa ggaattgacg ggggcccgca |
| 901 | caagcggagg aacatgtggt ttaattcgat gatacgcgag gaaccttacc cgggcttgaa |
| 961 | agttagtgac gattctggaa acaggatttc ccttcggggc acgaaactag gtgctgcatg |
| 1021 | gttgtcgtca gctcgtgccg tgaggtgtcg ggttaagtcc cataacgagc gcaaccccta |
| 1081 | ccgttagttg ccatcaggtc aagctgggca ctctggcggg actgccggtg taagccgaga |
| 1141 | ggaaggtggg gatgacgtca aatcagcacg gcccttacgt ccggggctac acacgtgtta |
| 1201 | caatggtagg tacagagggc cgctaccccg cgaggggatg ccaatctcga aagcctatct |
| 1261 | cagttcggat cggaggctga aacccgcctc cgtgaagttg gattcgctag taatcgcgca |
| 1321 | tcagccatgg cgcggtgaat acgttcccgg gccttgtaca caccgcccgt caagccatgg |
| 1381 | aagctggggg tgcctgaagt tcgtgaccgc aaggagcgac ctagggcaaa accggtgact |
| 1441 | ggggct |

The quantitative abundance is determined individually, from respective extracted DNA sequences, using a first set of probes and a second set of probes specific to each of the plurality of predetermined microbes associated with the saliva sample and the dental plaque sample respectively. The first set of probes includes a plurality of probes where each probe is utilized for each of the plurality of predetermined microbes (one probe for one predetermined microbe) associated with the saliva sample. Similarly, the second set of probes includes the plurality of probes where each probe is utilized for each of the plurality of predetermined microbes (one probe for one predetermined microbe) associated with the dental plaque sample.

In an embodiment, a multiplexed quantitative Polymerase Chain Reaction (qPCR) technique is employed for determining the quantitative abundance. More specifically, the multiplexed quantitative Polymerase Chain Reaction (qPCR) technique define a layout and arrangement of the plurality of probes that are of the first set of probes for determining the quantitative abundance associated with the saliva sample. Similarly, the multiplexed quantitative Polymerase Chain Reaction (qPCR) technique define the layout and arrangement of the plurality of probes that are of the second set of probes in the form of sequential runs for determining the quantitative abundance associated with the dental plaque sample.

More specifically, the first set of probes specific to each of the plurality of predetermined microbes associated with the saliva sample are utilized in two sequential multiplexed qPCR runs (defined by the multiplexed quantitative Polymerase Chain Reaction (qPCR) technique), to determine the quantitative abundance of each of the plurality of predetermined microbes associated with the saliva sample.

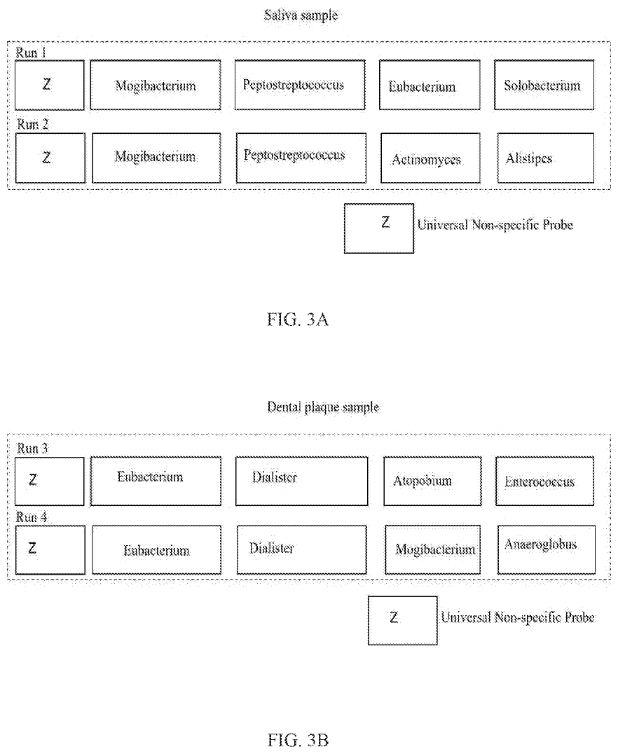

FIG. 3A illustrates an exemplary probe and multiplexed qPCR design for detecting and determining the quantitative abundance of each of the plurality of predetermined microbes associated with the saliva sample, according to some embodiments of the present disclosure. As shown in FIG. 3A, the two sequential multiplexed qPCR runs namely a first multiplexed qPCR run (Run 1) and a second multiplexed qPCR run (Run 2) are defined for determining quantitative abundance of each of the plurality of predetermined microbes associated with the saliva sample. Each Run of the first multiplexed qPCR run (Run 1) and the second multiplexed qPCR run (Run 2) includes five probes (hence it is also called as five-plex qPCR run) where each probe is utilized for the plurality of predetermined microbes associated with the saliva sample, from the list having: Mogibacterium, Peptostreptococcus, Eubacterium, Solobacterium, Actinomyces, and Alistipe. Also, each run of the first multiplexed qPCR run (Run 1) and the second multiplexed qPCR run (Run 2) contains a non-specific probe (denoted as ‘Z’ in FIG. 3A) at the start.

Further, as shown in FIG. 3A, the plurality of predetermined microbes, the quantitative abundance of which are being determined through the first multiplexed qPCR run are: Mogibacterium, Peptostreptococcus, Eubacterium, and Solobacterium. Further, the plurality of predetermined microbes, the quantitative abundance of which are being determined through the second multiplexed qPCR run are: Mogibacterium, Peptostreptococcus, Actinomyces, and Alistipes.

Similarly, the second set of probes specific to each of the plurality of predetermined microbes associated with the dental plaque sample are utilized in the two sequential multiplexed qPCR runs (defined by the multiplexed quantitative Polymerase Chain Reaction (qPCR) technique), to determine the quantitative abundance of each of the plurality of predetermined microbes associated with the dental plaque sample.

FIG. 3B illustrates an exemplary probe and multiplexed qPCR design for detecting and determining the quantitative abundance of each of the plurality of predetermined microbes associated with the dental plaque sample, according to some embodiments of the present disclosure. As shown in FIG. 3B, the two sequential multiplexed qPCR runs namely a third multiplexed qPCR run (Run 3) and a fourth multiplexed qPCR run (Run 4) are defined for determining quantitative abundance of each of the plurality of predetermined microbes associated with the dental plaque sample. Each Run of the third multiplexed qPCR run (Run 3) and the fourth multiplexed qPCR run (Run 4) includes five probes (hence it is also called as five-plex qPCR run) where each probe is utilized for the plurality of predetermined microbes associated with the dental plaque sample, from the list having: Eubacterium, Dialister, Atopobium, Enterococcus, Mogibacterium, and Anaeroglobus. Also, each run of the third multiplexed qPCR run (Run 3) and the fourth multiplexed qPCR run (Run 4) contains the non-specific probe (denoted as ‘Z’ in FIG. 3B) at the start.

Further, as shown in FIG. 3B, the plurality of predetermined microbes, the quantitative abundance of which are being determined through the third multiplexed qPCR run are: Eubacterium, Dialister, Atopobium, and Enterococcus. Further, the plurality of predetermined microbes, the quantitative abundance of which are being determined through the fourth multiplexed qPCR run are: Eubacterium, Dialister, Mogibacterium, and Anaeroglobus.

In an embodiment, the plurality of predetermined microbes associated with the saliva sample and the plurality of predetermined microbes associated with the dental plaque sample are captured from features of the respective pre-determined machine learning (ML) model. In an embodiment, the pre-determined machine learning (ML) model associated to the saliva sample is an ensemble machine learning (ML) model that is built using a microbial abundance data corresponding to a plurality of training saliva samples. The plurality of training saliva samples are the saliva samples used for training a machine learning model to obtain the corresponding pre-determined machine learning (ML) model. In an embodiment, the microbial abundance data corresponding to the plurality of training saliva samples is the quantitative abundance of all the microbes present in each of the plurality of training saliva samples.

Similarly, the pre-determined machine learning (ML) model associated to the dental plaque sample is the ensemble machine learning (ML) model that is built using the microbial abundance data corresponding to a plurality of training dental plaque samples. The plurality of training dental plaque samples are the dental plaque samples used for training the machine learning model to obtain the corresponding pre-determined machine learning (ML) model. In an embodiment, the microbial abundance data corresponding to the plurality of training dental plaque samples is the quantitative abundance of all the microbes present in each of the plurality of training dental plaque samples.

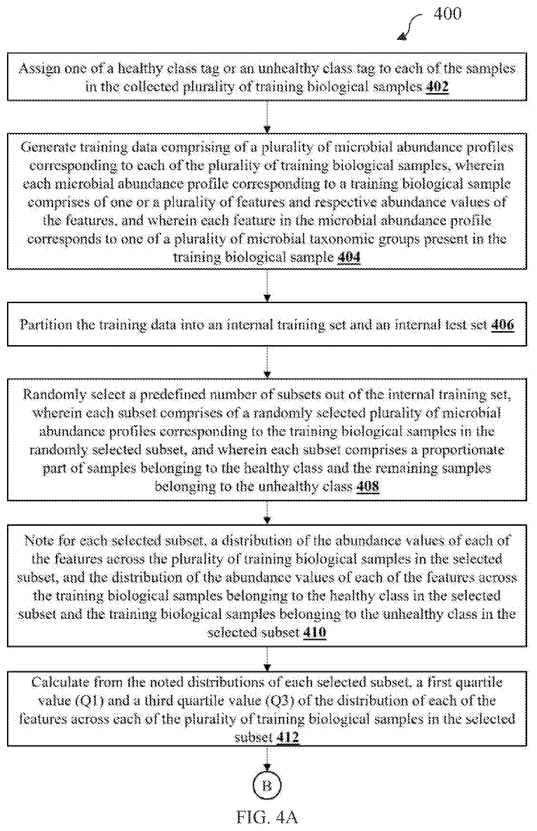

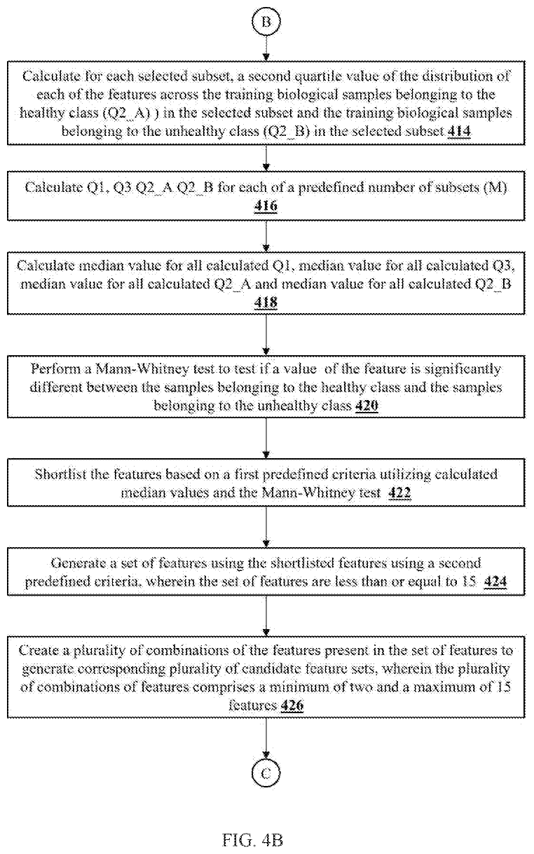

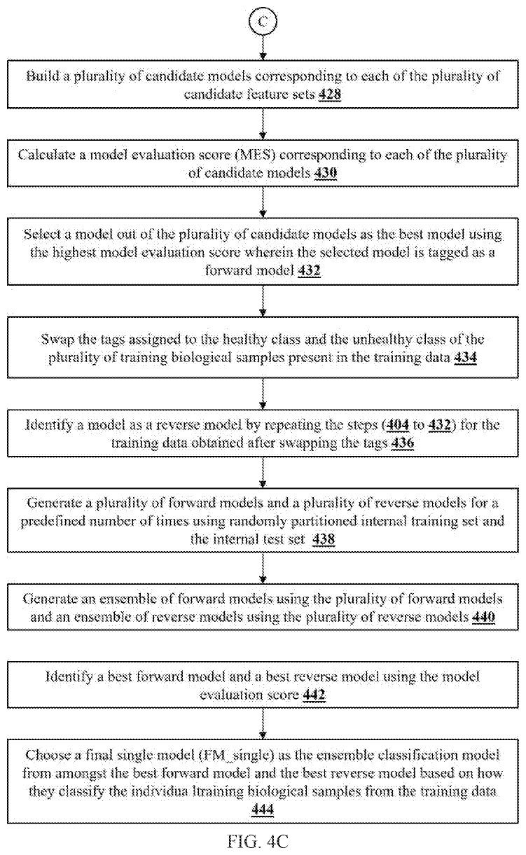

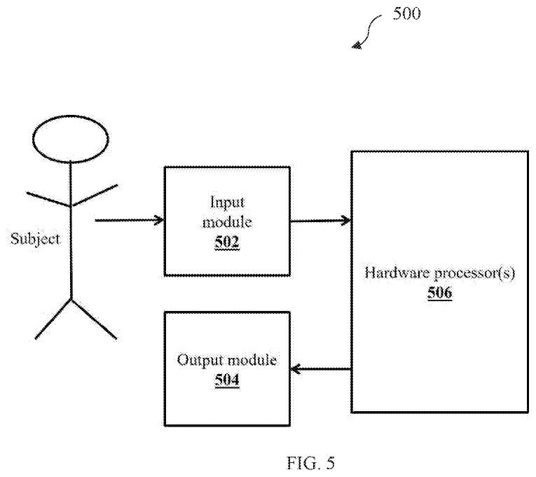

The ensemble ML model is built using the plurality of training biological samples consisting of saliva samples and dental plaque samples, individually, to obtain the corresponding pre-determined machine learning model. FIGS. 4A, 4B and 4C are flowcharts illustrating steps involved in building a pre-determined machine learning model according to some embodiments of the present disclosure. The technique for building the ensemble ML model accepts data in form of a feature table for multiple observations (the plurality of training biological samples) wherein each observation/training biological sample is defined by ‘N’ features (F) which are either or both of continuous and counted variables with (N≥1). In case of training data (TR), each of the training biological samples/observations further have a preassigned class/category which is binary in nature, i.e., a healthy class (A) (e.g., affiliating to biological samples sourced from healthy (neurotypical) individuals i.e., with no symptoms/history of ASD) and an unhealthy class or diseased class (B) (e.g., affiliating to biological samples sourced from individuals with symptoms of ASD or are diagnosed with ASD). In case of test data (TS) or data received during actual deployment of the method, the model(s) built based on training data predicts the class/category of the test biological samples/observations. During training process, the following steps are followed:

Initially at step 402, a healthy class tag or an unhealthy class tag is assigned to each of the training biological samples in the collected plurality of training biological samples (either saliva samples or dental plaque samples). The healthy class tag indicates samples sourced from healthy (neurotypical) individuals i.e., with no symptoms/history of ASD, and the unhealthy class tag indicates samples sourced from individuals with symptoms of ASD or are diagnosed with ASD.

At step 404, the training data comprises of a plurality of microbial abundance profiles corresponding to each of the collected plurality of training biological samples, wherein each microbial abundance profile corresponding to a training biological sample comprises of one or a plurality of feature (s) and respective abundance value (s) of the feature (s), wherein each feature in the microbial abundance profile corresponds to one of a plurality of microbial taxonomic groups present in the plurality of training biological samples.

In the next step 406, the training data (TR) is randomly partitioned into two sets—namely, an internal-train (ITR) and an internal-test (ITS), based on a parameter ‘L₁’, wherein L₁% training biological samples from the total training data constitute the ITR set and (100−L₁) % of the training biological samples constitute the ITS set. Furthermore, the random partitioning into ITR and ITS sets is performed using a stratified sampling approach with the intent of preserving the relative proportion of training biological samples belonging to the healthy class (A) or the unhealthy class (B) in the total training data in these newly drawn subsets.

In the next step 408, a predefined number of subsets are randomly selected out of the internal training set based on a second parameter (L₂). Each of the subset comprises a randomly selected plurality of microbial abundance profiles corresponding to the plurality of training biological samples in the randomly selected subset, and wherein each of the subset comprises a proportionate part of training biological samples belonging to the healthy class (A) and the remaining training biological samples belonging to the unhealthy class (B). Thus, from ITR, ‘M’ randomly drawn subsets ITRSᵢ (e.g., ITRS₁, ITRS₂, ITRS₃ . . . . ITRS_{M}), each containing S training biological samples are further generated, wherein S=L₂% of the training biological samples present in ITR. For example, the values of L₂ and M are 80% and 100 respectively for present disclosure. Other values are within the scope of this invention.

In the next step 410, for each selected subset, a distribution of the abundance values of each of the features across the plurality of training biological samples in the selected subset, and the distribution of the abundance values of each of the features across the training biological samples belonging to the healthy class (A) in the selected subset and the training biological samples belonging to the unhealthy class (B) in the selected subset are noted. Thus, from each subset ITRSᵢ(where i=1, 2, 3, . . . , M), wherein there are total S training biological samples, each of which are described by N features (Fⱼ) (where j=1, 2, 3, . . . , N), the distributions of each of the features (ITRSᵢDFⱼ) across S training biological samples are noted. Similarly, from each subset ITRSᵢ, wherein there are S_{A} training biological samples belonging to the healthy class (A) and S_{B} training biological samples belonging to the unhealthy class (B), each of the training biological samples being described by N features (Fⱼ; j=1, 2, 3, . . . , N), the distributions of each of the features (ITRSᵢD_{A}Fⱼ) across S_{A} training biological samples, and the distributions of each of the features (ITRSᵢD_{B}Fⱼ) across S_{B} training biological samples are noted.

In the next step 412, from the noted distributions of each selected subset, a first quartile value (Q1) and a third quartile value (Q3) of the distribution of each of the features is calculated across each of the plurality of training biological samples in the selected subset. In an example, the respective first quartile value (Q1) and the third quartile value (Q3) of ITRSᵢDFⱼ may also be referred as Q1ITRSᵢDFⱼ and Q3ITRSᵢDFⱼ.

Furthermore, in the next step 414, for each selected subset, a second quartile value of the distribution of each of the features across the training biological samples belonging to the healthy class (Q2_{A}) in the selected subset and the training biological samples belonging to the unhealthy class (Q2_{B}) in the selected subset is calculated. Thus, in an example, the median value (in other words, the second quartile value) of (ITRSᵢD_{A}Fⱼ) is referred as Q2ITRSᵢD_{A}Fⱼ, and the median value of (ITRSᵢD_{B}Fⱼ) is referred as Q2ITRSᵢD_{B}Fⱼ.

In the next step 416, for the M subsets of ITRSⱼ, a total of M values for each of Q1ITRSᵢDFⱼ, Q3ITRSᵢDFⱼ, Q2ITRSᵢD_{A}Fⱼ, and Q2ITRSᵢD_{B}Fⱼ, are calculated. Further at step 418, median value ( [symbol] ⱼ) is calculated for all calculated Q1, median value ( [symbol] ⱼ) is calculated for all calculated Q3, median value ( [symbol] ) is calculated for all calculated Q2_{A} and median value ( [symbol] ) is calculated for all calculated Q2_{B}. Thus,

$${J}_{} = median of \{ Q 1 {ITRS}_{1} {DF}_{j} , Q 1 {ITRS}_{2} {DF}_{j} , Q 1 {ITRS}_{3} {DF}_{j} , … Q 1 {ITRS}_{M} {DF}_{j} \} \\ {J}_{} = median of \{ Q 3 {ITRS}_{1} {DF}_{j} , Q 3 {ITRS}_{2} {DF}_{j} , Q 3 {ITRS}_{3} {DF}_{j} , … Q 3 {ITRS}_{M} {DF}_{j} \} \\ = median of \{ Q 2 {ITRS}_{1} {D}_{A} {F}_{j} , Q 2 {ITRS}_{2} {D}_{A} {F}_{j} , Q 2 {ITRS}_{3} {D}_{A} {F}_{j} , … Q 2 {ITRS}_{M} {D}_{A} {F}_{j} \} \\ = median of \{ Q 2 {ITRS}_{1} {D}_{B} {F}_{j} , Q 2 {ITRS}_{2} {D}_{B} {F}_{j} , Q 2 {ITRS}_{3} {D}_{B} {F}_{j} , … Q 2 {ITRS}_{M} {D}_{B} {F}_{j} \}$$

$$( where i = 1 , 2 , 3 , … , M ; and j = 1 , 2 , 3 , … , N \}$$

In the next step 420, a Mann-Whitney test is performed to test if a value of the feature (Fⱼ) is significantly (p<0.1) different between the training biological samples belonging to the healthy class (S_{A}) and the training biological samples belonging to the unhealthy class (S_{B}) in each of the M randomly drawn subsets ITRSⱼ. Other statistical tests based on the nature of distribution (e.g., t-test for normal distribution), nature of sampling (e.g., Wilcoxon signed rank test for paired case and control samples) or other methods of statistical comparison relevant for microbiome datasets (e.g., ALDEx2) can also be adopted.

In the next step 422, the features are shortlisted based on a first predefined criteria utilizing calculated median values and the Mann-Whitney test. The first predefined criteria comprises if a feature Fⱼ is observed to have significantly (p<0.1) different values in S_{A} compared to S_{B} in more than 70% of M subsets, and if [symbol] >=Q2ₘᵢₙ OR [symbol] >=Q2ₘᵢₙ (a predefined feature ‘abundance’ threshold and Q2ₘᵢₙ threshold as described in the case study). Fⱼ is added to a set of shortlisted features (SF).

In the next step 424, a set of features is generated using the shortlisted features (SF) using a second predefined criteria, wherein the set of features are less than or equal to 15. If the number of shortlisted features (SF) obtained in previous step satisfies the criteria 1≤SF≤15, then the training process proceeds to model building with all the features in SF. If no shortlisted features (SF) are obtained in previous (i.e., SF<1) then following step is performed with all the features Fⱼ for evaluating the ability of the features, when considered independently, to distinguish between training biological samples belonging to the healthy class (A) and the unhealthy class (B). Similarly, if the number of shortlisted features (SF) obtained in previous step exceeds fifteen (SF>15) then following step is performed with all the shortlisted features (SF) for evaluating the ability of the features, when considered independently, to distinguish between the training biological samples belonging to the healthy class (A) and the unhealthy class (B).

Steps for shortlisting the features in case of SF<1 or SF>15: For each of the features (obtained previously) taken individually, different threshold values are used to classify the samples belonging to the set ITR, and the results are cumulated to construct a receiver operating characteristic curve (ROC curve) for each of the features. The area under the curve (AUC) of the ROC curve of any feature (AUC^{F}) is indicative of the utility of the feature to distinguish between the training biological samples belonging to the healthy class (A) and the unhealthy class (B), and the same is computed for every feature. The shortlisted features (SF) set is modified to include only the top fifteen features from a list of features arranged in a descending order of the AUC^{F} values.

In the next step 426, a plurality of combinations of the features present in the set of features is created to generate corresponding plurality of candidate feature sets (CF), wherein the plurality of combinations of features comprises a minimum of one and a maximum of 15 features. In an embodiment, the maximum possible candidate feature sets that can be created in this process is K=2¹⁵−1=32767 (i.e., maximum value of K=32767).

In the next step 428, a plurality of candidate models is built corresponding to each of the plurality of candidate feature sets. At step 430, a model evaluation score (MES) is calculated corresponding to each of the plurality of candidate models. For each candidate feature set CF_{K}, a corresponding candidate model CM_{K} is built and evaluated as mentioned in the steps mentioned below.

### Steps for Evaluating the Candidate Model:

Step 1: The values of the features F constituting a candidate feature set defining the training biological samples in ITR are transformed to Fⱼ′ such that − [symbol] ⱼ, [symbol] ⱼ, [symbol] and

[symbol]

$${F}_{j}^{′} = 0 if {F}_{j} < {J}_{} \\ {F}_{j}^{′} = 1 if {F}_{j} > {\tilde{Q 3}}_{J} \\ {F}_{j}^{′} = 0.5 if {\tilde{Q 1}}_{J} = {\tilde{Q 3}}_{J} \\ {F}_{j}^{′} = \frac{{F}_{j} - {\tilde{Q 1}}_{J}}{{\tilde{Q 3}}_{J} - {( \tilde{Q 1} )}_{J}} if {\tilde{Q 1}}_{J} < {F}_{j} <$$

Step 2: If for a feature Fⱼ, it is observed that [symbol] > [symbol] then the feature Fⱼ is tagged as a ‘numerator’ feature and added to a set of numerator features Fₙᵤₘₑᵣₐₜₒᵣ. Else, feature Fⱼ is tagged as a ‘denominator’ feature and added to a set of denominator features F_{denominator}.
Step 3: Each candidate model (CM_{K}) is constituted as a simple ratio function given below—

$${CM}_{K} = \frac{Σ {F}_{numerator}}{Σ {F}_{denominator}} when {F}_{numerator} > 0 and {F}_{denominator} > 0 or , \\ {CM}_{K} = \frac{Σ {F}_{numerator} + 1}{Σ {F}_{denominator} + 1} when either {F}_{numerator} or {F}_{denominator} = 0$$

wherein, ΣFₙᵤₘₑᵣₐₜₒᵣ represents the sum of values of all numerator features for a particular sample, and,
wherein, ΣF_{denominator} represents the sum of values of all denominator features for a particular sample.

For each of the features, a transformed value Fⱼ′ as obtained above is used in the candidate model equation.
Step 4: A candidate model c is used to generate candidate model scores (CMS_{K}) for each of the samples in the set ITR. From the set of scores CMS_{K}, the top 10 percentile and bottom 10 percentile scores are removed as outliers and thereafter the maximum and minimum scores from the set CMS_{K} are noted as CMS_{K}_{ₘₐₓ} and CMS_{K}_{ₘᵢₙ} respectively.
Step 5: Considering each of the scores in the set CMS_{K} as a threshold (T), the model CM_{K} is used to (re)classify the samples in the training set (ITR) such that—
   the training biological sample is classified into the healthy class (A) if CMS>=T
   or the training biological sample is classified into the unhealthy class (B) if CMS<T

and based on a comparison of these classifications and the true/original classes of the training biological samples, Matthew's correlation coefficients (MCC) for each of the thresholds are calculated, to evaluate how well each of the thresholds can distinguish between training biological samples between the healthy class (A) and the unhealthy class (B).
Step 6: The threshold (Tₘₐₓ) which provides the maximum absolute MCC value (|MCCₘₐₓ|) is noted. If |MCCₘₐₓ|<0.4 for a candidate model CM_{K}, then the candidate model is discarded from further evaluation. Else, the |MCCₘₐₓ| value is considered as the ‘train-MCC’ value (|MCCₜᵣₐᵢₙ|) for the model ITS and the model and its corresponding Tₘₐₓ threshold is used to classify the training biological samples in the internal-test set (ITS). In another implementation of the process, the MCCₘₐₓ threshold may not be applied for retaining the candidate model for subsequent evaluation. Before classifying the each of the training biological samples in the ITS set, the values of features characterizing the training biological samples of the ITS set are transformed using the method mentioned in step 418 while using the earlier obtained values of [symbol] ⱼ, [symbol] ⱼ, [symbol] and [symbol] from the ITR set.
Step 7: The classification results on the training biological samples from the ITS set are compared against the true/original classes of the training biological samples (with pre-assigned labels), and the MCC for the model CM_{K} and its corresponding Tₘₐₓ threshold on the ITS samples is calculated (MCCₜₑₛₜ).
Step 8: A model evaluation score (MES) for candidate model CM_{K} is calculated as MES=|(MCCₜᵣₐᵢₙ+MCCₜₑₛₜ)|−|(MCCₜᵣₐᵢₙ−MCCₜₑₛₜ)|

In the next step 432, the model CM_{K} is tagged as a “strong model” if all the features in the corresponding candidate feature set satisfies the Mann-Whitney test based shortlisting criteria described above. Otherwise, if any of the features in the corresponding feature set fails to satisfy the Mann-Whitney test, the model CM_{K} is tagged as a “weak model”.

Further, the above process is repeated for candidate models and respective MES scores are used to rank all the models. The best model is subsequently chosen based on the MES score. In case there are more than one model with the best MES score, the best model is chosen based on the following criteria (in order of preference):
(a) the model with fewer number of features (i.e., based on a smaller candidate feature set) is chosen.
(b) the model with lower Tₘₐₓ (threshold value) is chosen.

Further, the best model obtained through above steps is tagged as a forward model (MD_{fwd}). The model MD_{fwd} additionally constitutes its corresponding Tₘₐₓ threshold, the CMS_{K}_{ₘₐₓ} and CMS_{K}_{ₘᵢₙ} values, and the [symbol] ⱼ, [symbol] ⱼ, [symbol] and [symbol] , values corresponding to the ITR set.

In the next step 434, the tags assigned to the healthy class (A) and the unhealthy class (B) of the plurality of samples present in the training data are swapped. At step 436, all of the above steps 404 to 432 to determine the best model are repeated after swapping the class labels (A<->B) for the entire training set (TR) to obtain a best model tagged as the reverse model (MDᵣₑᵥ). The model (MDᵣₑᵥ) additionally constitutes its corresponding Tₘₐₓ threshold, the CMS_{K}_{ₘₐₓ} and CMS_{K}_{ₘᵢₙ} values, and the [symbol] ⱼ, [symbol] ⱼ, [symbol] , and [symbol] values corresponding to the ITR set.

At step 438, a plurality of forward models and a plurality of reverse models are generated by repeating step (404) through (436) for a predefined number of times using randomly partitioned internal training set and the internal test set. The steps (404) through (436) are iterated ‘R’ times using multiple randomly partitioned ITR and ITS sets generated initially. After each iteration, (i) the features constituting the models MD_{fwd} and the models MDᵣₑᵥ obtained in the current iteration (r) are compared against, and if necessary, appended to, a set of unique features F_{unq} that consists of respective features constituting the MD_{fwd} and MDᵣₑᵥ obtained in earlier iterations (i.e., up to iteration r−1). After ‘R’ iterations, a plurality of forward models and a plurality of reverse models are generated for a predefined number of times using randomly partitioned internal training set and the internal test set. The iterations proceed while the value of R satisfies the following criteria—
(i) R≤Rₘₐₓ
(ii) (|F_{unq}| after iteration R)>(|F_{unq}| after iteration R−R_{unq})
(iii) |F_{unq}| after iteration no. R<=Fetₘₐₓ
   Wherein, Rₘₐₓ is a parameter indicating the maximum number of iterations allowed;
   R_{unq} is a parameter indicating the maximum number of iterations allowed without any cumulative increase in the number of unique features |F_{unq}| in the models being generated in consecutive iterations; and
   Fetₘₐₓ is a parameter indicating the maximum allowed value of |F_{unq}| (i.e., the no. of unique features cumulated through the iterative process).

In an embodiment, the exemplary values of Rₘₐₓ, R_{unq}, and Fetₘₐₓ are 100, 10, 100 respectively for the present disclosure. Other values of these and other parameters here for finetuning and suitability for other datasets are within the scope of the present invention.

In the next step at 440, an ensemble of forward models is generated using the plurality of forward models and an ensemble of reverse models is generated using the plurality of reverse models. This is referred as an ensemble of forward models (ENS-MD_{fwd})) and an ensemble of reverse models (ENS-MDᵣₑᵥ).

At step 442, the best models from each of these ensembles, i.e., the best of the forward models (BMD_{fwd}) and the best of the reverse models (BMDᵣₑᵥ) respectively, are identified.

If all models in an ensemble are weak models, the best model from the ensemble (BMD) is chosen by ranking the models based on their model evaluation scores and associated criteria. Also, if an ensemble contains more than one strong model, then only those strong models are considered for ranking based on their model evaluation scores and associated criteria as mentioned above, and the best model from the ensemble (BMD) is thereby chosen.

In the next step 444, a final single model (FM_{single}) is chosen as the ensemble classification model from amongst the best forward model and the best reverse model based on how they classify the individual samples from the training data. Once the best models from each of the ensemble of forward models and the ensemble of reverse models, i.e., the best of the forward models (BMD_{fwd}) and the best of the reverse models (BMDᵣₑᵥ) are identified, the final single model (FM_{single}) is chosen from amongst BMD_{fwd} and BMDᵣₑᵥ based on how well they can classify the individual training biological samples from the entire training set (TR). The AUC value for ROC curves for each of these two models are computed based on the predicted model scores for the training set (TR) samples and their pre-assigned classes (the healthy class (A) and the unhealthy class (B)). The model having the best AUC for ROC value is selected as the final single model (FM_{single}). If both BMD_{fwd} and BMDᵣₑᵥ have the same AUC value, BMD_{fwd} is chosen as FM_{single}.

In an alternate implementation FM_{single} can be chosen based whether BMD_{fwd} or BMDᵣₑᵥ obtains a higher MCC value while classifying the TR training biological samples. Once the FM_{single} model has been chosen, for classification of any samples from a test set (TS) or any sample data received during actual deployment, the FM_{single} model is used after:
(a) appropriately transforming the features corresponding to the training biological sample being classified using the [symbol] ⱼ, [symbol] ⱼ, [symbol] and [symbol] values corresponding to the FM_{single} model,
(b) limiting the model score between a maximum of CMS_{K}_{ₘₐₓ} and a minimum of CMS_{K}_{ₘᵢₙ} values corresponding to the FM_{single} model, and
(c) classification based on the model score using its corresponding threshold Tₘₐₓ.

According to an embodiment of the disclosure, the ensemble of forward models (ENS-MD_{fwd}) and the ensemble of reverse models (ENS-MDᵣₑᵥ) are also evaluated for their collective classification efficiencies using an ensemble model scoring. In the ensemble scoring method, each of the models (MD) constituting an ensemble (ENS) are used to generate a model score (MS) for each of the samples from the entire TR set. For any specific training biological sample, the values of the features corresponding to the training biological sample are appropriately transformed using the [symbol] ⱼ, [symbol] ⱼ, [symbol] and [symbol] values corresponding to the model MD. The model scores (MS) are then transformed into scaled model scores (SMS) having values between −1 and +1, using the following procedure:

$$SMS = ( MS - {T}_{max} ) / ( {CMS}_{{K}_{max}} - {T}_{max} ) , when MS >= {T}_{max} , and \\ SMS = ( MS - {T}_{max} ) / ( {T}_{max} - {CMS}_{{K}_{min}} ) , when MS < {T}_{max} ,$$

Wherein, Tₘₐₓ, CMS_{K}_{ₘₐₓ}, and CMS_{K}_{ₘᵢₙ} values corresponding to the respective model is used.

Let SMS_{avg} be the average of all SMS obtained using all models in ENS for a particular sample.

When using Forward model [ENS-MD_{fwd}],

$${SMS}_{avg} = {SMS}_{avg} * ( + 1 )$$

If SMS_{avg}>=0, sample is classified as the unhealthy class (B)
If SMSₐ˜g<0, sample is classified as the healthy class (A)
   When using Reverse model [ENS-MDᵣₑᵥ]:

$${SMS}_{avg} = {SMS}_{avg} * ( - 1 )$$

If SMS_{avg}>0, sample is classified as the unhealthy class (B)
If SMS_{avg}<=0, sample is classified as the healthy class (A)

If all models in one of the ensembles are weak models, then the other one having (one or more) strong models is selected as a final ensemble model (FMₑₙₛ), and subsequently used for classification of any of training biological samples from a test set (TS) or any sample data received during actual deployment of the method, using the scoring and classification process mentioned in above paragraph. If both ensembles have constituent strong models, then both the ensembles are evaluated for their efficiency by scoring them on all individual samples in TR. The AUC value for ROC curves for each of these two ensembles are computed based on the predicted SMS_{avg} for all the training set (TR) samples and their pre-assigned classes. The ensemble of models having the best AUC for ROC value is selected as the final ensemble model (FMₑₙₛ). In case both ENS-MD_{fwd} and ENS-MDᵣₑᵥ exhibit equal AUC values then ENS-MD_{fwd} is chosen as the final ensemble model (FMₑₙₛ). In an alternate implementation, FMₑₙₛ can be chosen based whether ENS-MD_{fwd} and ENS-MDᵣₑᵥ obtains a higher average MCC value for their respective constituent models while classifying the TR samples.

Thus, either the FM_{single} model or FMₑₙₛ ensemble of models can be used for classification of any of training biological samples from a test set (TS) or any training biological sample data received during actual deployment.

In an embodiment, one or more predetermined microbes out of the plurality of predetermined microbes associated with the saliva sample, are common to the first multiplexed qPCR run (Run 1) and the second multiplexed qPCR run (Run 2) for determining the associated quantitative abundance. In an embodiment, the one or more predetermined microbes that are common to the first multiplexed qPCR run (Run 1) and the second multiplexed qPCR run (Run 2) are determined based on (i) a median abundance (obtained from microbial abundance data) of each of the plurality of predetermined microbes obtained from the plurality of training saliva samples, (ii) a frequency of occurrence of each of the plurality of predetermined microbes constituting the ensemble ML model associated with the saliva sample. More specifically, the one or more predetermined microbes (from amongst the set of predetermined microbes) has/have the highest (or relatively higher) median abundance or frequency of occurrence (as compared to the median abundance(s) or the frequency of occurrence of each microbe in the remaining set of predetermined microbes) across the plurality of training saliva samples is/are common to the first multiplexed qPCR run (Run 1) and the second multiplexed qPCR run (Run 2).

For example, a predetermined microbe having a high median abundance or a high frequency of occurrence from the microbial abundance data is determined and utilized in more than one Run. As shown in FIG. 3A, the predetermined microbe Mogibacterium is common for both the first multiplexed qPCR run (Run 1) and the second multiplexed qPCR run (Run 2). Similarly, the predetermined microbe Peptostreptococcus is also common for both the first multiplexed qPCR run (Run 1) and the second multiplexed qPCR run (Run 2).

Similarly, the one or more predetermined microbes out of the plurality of predetermined microbes associated with the dental sample, are common to the third multiplexed qPCR run (Run 3) and the fourth multiplexed qPCR run (Run 4) for determining the associated quantitative abundance. In an embodiment, the one or more predetermined microbes that are common to the third multiplexed qPCR run (Run 3) and the fourth multiplexed qPCR run (Run 4) are determined based on (i) the median abundance (obtained from microbial abundance data) of each of the plurality of predetermined microbes obtained from the plurality of training dental plaque samples, (ii) the frequency of occurrence of each of the plurality of predetermined microbes constituting the ensemble ML model associated with the dental plaque sample. More specifically, the one or more predetermined microbes (from amongst the set of predetermined microbes) has/have the highest (or relatively higher) median abundance or frequency of occurrence (as compared to the median abundance(s) or the frequency of occurrence of each microbe in the remaining set of predetermined microbes) across the plurality of training dental plaque samples is/are common to the third multiplexed qPCR run (Run 3) and the fourth multiplexed qPCR run (Run 4).

For example, a predetermined microbe having a high median abundance or a high frequency of occurrence from the microbial abundance data is determined and utilized in more than one Run. As shown in FIG. 3B, the predetermined microbe Eubacterium is common for both the third multiplexed qPCR run (Run 3) and the fourth multiplexed qPCR run (Run 4). Similarly, the predetermined microbe Dialister is also common for both the third multiplexed qPCR run (Run 3) and the fourth multiplexed qPCR run (Run 4).

In an embodiment, the quantitative abundance determination involves creating abundance or feature table and generation of the percent normalized abundance or feature table having percent normalized abundance values of the predetermined microbes or OTUs or taxas in each sample. In another embodiment, Multicolour Combinatorial Probe Coding (MCPC) qPCR or real-time PCR based measurement of abundance of the microbial OTUs or taxas can also be considered for quantification of a predefined set of taxas. Alternatively, any other pre-processing techniques or data normalization techniques known in the state of art can be used for normalization and feature selection from the main feature table.

Design Configuration & Number of Multiplexed qPCR Runs Required for Quantifying the Abundance of Target Microbes or Microbial Taxonomic Groups or Microbial Taxa/Features:

The quantitative abundance of each of the microbial taxonomic groups or microbes, that are common to each of the multiplexed qPCR runs (the first multiplexed qPCR run, and the second multiplexed qPCR run), is determined based on a normalizing factor (NFᵣᵤₙ) associated with each multiplexed qPCR run and the quantitative abundance of associated microbial taxonomic group in the corresponding multiplexed qPCR run.

For example, considering a maximum of five unique DNA fragments, each representing a microbial taxa or spike DNA, can be quantified in a one multiplexed qPCR run. Therefore, to analyze a disease signature (captured in an ML model) comprising of ‘n’ microbial taxa/features, a minimum of (1+┐(n−4)/4┌) multiplexed qPCR runs would be required wherein ‘n’ is the unique number of microbial taxonomic groups constituting the frugal set of markers, and wherein each multiplexed qPCR run is configured to determine, in the test biological sample, the relative abundance of a predetermined subset of the microbial taxonomic groups constituting the disease signature. This minimum number is based on assumptions that:
(a) the spike DNA should be analyzed at least once in one of the ‘(1+┌(n−4)/4┐)’ multiplexed qPCR runs; and
(b) an overlap of at least one microbial taxa/features was done between two corresponding runs.

For example, if a disease signature comprises of 8 microbial taxa (A, B, C, D, E, F, G, and H), then at least TWO multiplexed qPCR runs would be required, where Z is the spike DNA of known concentration and taxa ‘D’ is analyzed in both multiplexed qPCR runs. Here, ┌(n−4)/4┐ indicates a ceiling value of the expression. Thus, the minimum no. of required qPCR runs would be:
1 for 1-4 signatures/features
2 for 5-8 signatures/features
3 for 9-12 signatures/features
4 for 13-16 signatures/features, and so on . . . .

### Example A: Run 1: Z A B C D; Run2: D E F G H

Similarly, for a feature size of 12 (A, B, C, D, E, F, G, H, I, J, K, and L), at least THREE multiplexed qPCR runs would be required, where Z is the spike DNA of known concentration and taxa ‘D’ and ‘H’ are analyzed in twice.

### Example B: Run 1: Z A B C D; Run2: D E F G H; Run3: H I J K L

If the number of features constituting the signature is not optimal for the above condition, i.e., for e.g., the number of features is 10, then more than one microbial taxon can be analyzed twice. The same is exemplified below, wherein taxa C and D are analyzed twice (in Runs 1 and 2). Similarly, taxa F and G are also analyzed twice (in Runs 2 and 3).

### Example C: Run 1: Z A B C D; Run 2: C D E F G; Run3: F G H I J

In alternate implementations, the spike DNA (Z) can be analyzed in each of the runs. In that scenario, the first multiplexed qPCR will be able to accommodate up to FOUR features. Each additional multiplexed qPCR run will accommodate up to THREE new/additional features as shown by underlining in the example below. Thus, two multiplexed qPCR runs would be required for a feature set of up to seven; three qPCR runs for a feature set of up to ten and so on.

### Run 1: Z A B C D; Run 2: Z D E F G; Run3: Z G H I J

Furthermore, if the number of features is not optimal for the above condition, then two or more taxa/features can be analyzed multiple times as shown in example C.

Methodology to Interpret/Quantify the Abundance of a Microbial Taxon or Microbes or Microbial Taxonomic Groups from Data Obtained from Above Qpcr Configurations:

Given that the concentration of the spike DNA (Z) is previously known—say X₁. If the measured concentration of Z in the multiplexed qPCR is X₂, then all the measured concentration in a single multiplexed qPCR run can be normalized multiplying by a normalizing factor (NFᵣᵤₙ) of X₁/X₂.

In cases where the spike DNA is only analyzed in only one of the multiplexed qPCR runs (as shown in examples A, B and C), then the normalized values of the taxa/feature in the first run which is/are re-analyzed in the Run 2, can be used for adjusting the concentrations inferred from the Run 2 of the multiplexed qPCR. Following Example-A (described previously),
Actual conc of Z: X₁
Measured conc of Z: X₂

$$Normalizing factor {NF}_{run 1} : {X}_{1} / {X}_{2} \\ Inferred conc . of A ( from Run 1 ) : {A}_{run 1}^{′} \times {NF}_{run 1} \\ Inferred conc . of B ( from Run 1 ) : {B}_{run 1}^{′} \times {NF}_{run 1} \\ Inferred conc . of C ( from Run 1 ) : {C}_{run 1}^{′} \times {NF}_{run 1} \\ Inferred conc . of D ( from Run 1 ) : {D}_{run 1}^{′} \times {NF}_{run 1}$$

Where A′ᵣᵤₙ₁, B′ᵣᵤₙ₁, C′ᵣᵤₙ₁, and D′ᵣᵤₙ₁ are the measured/analyzed concentrations of taxa/feature A, B, C and D respectively.

Normalizing factor NFᵣᵤₙ₂: Inferred conc. of D from Run 1/Measured concentrations of feature D in Run 2

$$Inferred conc . of E : {E}_{run 1}^{′} \times {NF}_{run 2} \\ Inferred conc . of F : {F}_{run 1}^{′} \times {NF}_{run 2} \\ Inferred conc . of G : {G}_{run 1}^{′} \times {NF}_{run 2} \\ Inferred conc . of H : {H}_{run 1}^{′} \times {NF}_{run 2}$$

The same protocol may be repeated for normalizing/adjusting the concentrations measured from all subsequent runs (as in example B). In case wherein more than once feature is analyzed in subsequent runs (as in example C), a median Normalizing factor (NF)—derived from the NFs for each of the replication features may be used for computing the inferred concentrations from that run.

In alternate implementations, wherein the spike DNA (Z) is analyzed in each of the runs (as in example D), Normalizing factor (NF) corresponding to each of the runs may be computed and used for inferring the concentrations of the constituent features. In cases, where the measured spike DNA (Z) concentration varies by more than 25% from the actual concentration, it is suggested that the observations from the said multiplexed qPCR run be discarded, and a fresh multiplexed qPCR run for the sub-set of features be performed.

In an alternate implementation using multiplexed qPCR runs, the marker feature (marker microbe or taxa) having the lowest variance in relative abundance in training data across both the classes, is selected as the anchor marker (AM), and the relative abundance of each of the markers is computed by multiplying the ratio of their estimated/inferred DNA concentrations and the estimated/inferred DNA concentration of AM with the median abundance of AM across all training data. For example, if the marker features are A, B, C and D. wherein A is the anchor marker (AM) having a median abundance of ABN_{AM}, then the abundances of the marker features B, C and D will be computed as;

$${ABN}_{B} = ( Inferred conc . of B / Inferred conc . of A ) \times {ABN}_{AM} \\ {ABN}_{C} = ( Inferred conc . of C / Inferred conc . of A ) \times {ABN}_{AM} \\ {ABN}_{D} = ( Inferred conc . of D / Inferred conc . of A ) \times {ABN}_{AM}$$

At step 208 of the method 200, the quantitative abundance is collated through the abundance determining module 112. The quantitative abundance of (i) each of the plurality of predetermined microbes associated with the saliva sample and (ii) each of the plurality of predetermined microbes associated with the dental plaque sample, determined at step 206 of the method 200 is collated to obtain a hybrid abundance matrix.

At step 210 of the method 200, a model score is determined based on the hybrid abundance matrix obtained at step 208 of the method 200, through the ML module 114. The ML module 114 includes a pre-determined machine learning (ML) model explained and obtained at step 206 of the method 200 is employed to determine the model score based on the hybrid abundance matrix.

At step 212 of the method 200, the risk assessment of the subject is performed through the assessment module 116. The risk assessment is performed based on the model score obtained at step 210 of the method 200 and a predefined threshold value. For example, if the model score obtained at step 210 of the method 200 is greater than or equal to the predefined threshold value, then the subject is assessed to be having the autism spectrum disorder. If the model score obtained at step 210 of the method 200 is less than the predefined threshold value, then the subject is assessed as not having the autism spectrum disorder.

At step 214 of the method 200, a personalized recommendation for the subject assessed as having autism spectrum disorder at step 212 of the method 200 is designed through the recommendation module 118. In an embodiment, the personalized recommendation includes utilizing a set of rules for the set of microbes that constitute the pre-determined machine learning model to identify one or more personalized probiotic and antibiotic candidates that may be employed to ameliorate disease symptoms in the subject identified as having autism spectrum disorder. In an embodiment, the microbes (organisms) contributing to generation of model score at step 210 are mapped to a predefined set of antibiotic and probiotic candidates and appropriate personalized targets for treatment and recommendation are identified accordingly.

More specifically, one or a combination of probiotic and antibiotic (microbial) candidates or targets designed via or based on features (i.e., taxa or microbes) constituting the ML model. The designing of the one or the combination of probiotic and antibiotic candidates' targets may be performed by mapping the features (i.e., organisms/taxa/microbes) constituting the ML model to the complete set of microbes (or a pre-defined subset of the same) using the following steps

At step 1, pair-wise correlations (using the Pearson's and/or spearman's correlation index) are computed between abundances of features (i.e., organisms/taxa/microbes) constituting the ML model and the abundances corresponding to the complete set of microbes computed individually from (a) the subset of biological samples corresponding to the healthy class i.e. the class of samples that are taken from individuals not having ASD, and (b) the diseased class i.e. the class of samples that are taken from individuals having ASD). Wherein the samples belonging to the healthy and diseased classes are used as training data for generating the ML model.

At step 2, positive and negative interactions between features (i.e., organisms/taxa/microbes) constituting the ML model and all other taxa in the healthy and the diseased class of training samples (individually) are deduced using critical correlation (r) value as the cut-off (as taught in Batushansky et al., 2016), such that inter-taxa correlation index values greater than +r value are affiliated as ‘positive interactions’, while those less than −r value are affiliated as ‘negative interactions’.

At step 3, the steps 1 and 2 are repeated 1000 times and only those interactions are considered relevant that appear in at least 70% of iterations with a BH (Benjamini-Hochberg) corrected p-value cut-off of 0.1 are retained (hereafter referred to as model taxa interactions corresponding to health and diseased class of samples).

At step 4, thereafter, following set of rules (indicated in Table 1 below) are used to arrive at the relevant candidate using the retained model taxa interactions:

TABLE 1

| Probiotic Candidates | Antibiotic Candidates |
|---|---|
| (M_{H} − C_{T})_{HP} && (M_{H} − C_{T})_{DP} | (M_{H} − C_{A})_{HN} \|\| (M_{H} − C_{A})_{DN} |
| (M_{H} − C_{T})_{DP} | (M_{D} − C_{A})_{HP} \|\| (M_{D} − C_{A})_{DP} |
| (M_{D} − C_{T})_{HN} && (M_{D} − C_{T})_{DN} | |

From Table 1,
M_{H} represents a model taxon having significantly higher abundance in healthy class;
M_{D} represents a model taxon having significantly higher abundance in diseased (unhealthy) class;
C_{T} represents a potential candidate for recommendation;
C_{A} represents a potential antibiotic target candidate;
M_{H}-C_{T} represents an interaction between a model taxon (abundant in healthy class) with a potential candidate for recommendation;
M_{D}-C_{T} represents an interaction between a model taxon (abundant in diseased class) with a potential candidate for recommendation;
M_{D}-C_{A} represents an interaction between a model taxon (abundant in diseased class) with a potential antibiotic target candidate;
M_{H}-C_{A} represents an interaction between a model taxon (abundant in healthy class) with a potential antibiotic target candidate;
HP represents a positive interaction in a healthy environment population;
HN represents a negative interaction in a healthy environment population;
DP represents a positive interaction in a diseased environment population; and
DN represents a negative interaction in a diseased environment population.

One or more of the set of microbes constituting the identified probiotic candidates may be recommended (individually or in combination) as probiotic formulations for treating (or ameliorating the symptoms of or the disease severity of) individuals identified as having ASD. Furthermore, the mentioned probiotic formulations may help in promoting development of a healthy oral microbiome (in the individuals administered with the probiotic) which may be employed to ameliorate the symptoms of, or the disease severity of individuals identified as having ASD.

One or more of the set of microbes constituting the identified antibiotic microbial candidates may be targeted (individually or in combination) via antibiotics or other treatment methodologies that can reduce the abundance of the identified antibiotic microbial candidate(s) and may be recommended for ameliorating the symptoms of or the disease severity of individuals identified as having ASD. Furthermore, such antibiotic recommendation (as detailed above) may also help in promoting development of a healthy oral microbiome, which (may) ameliorate the symptoms of, or the disease severity of individuals identified as having ASD.

Further, a kit for risk assessment of autism spectrum disorder in the subject, is disclosed. FIG. 5 illustrates an exemplary block diagram of a kit 500 for risk assessment of autism spectrum disorder present in the subject, according to some embodiments of the present disclosure. As shown in FIG. 5, the kit 500 includes an input module 502, one or more hardware processors 504 and an output module 506. The input module 502 is used for receiving the saliva sample and the dental plaque sample of the subject whose risk of autism spectrum disorder is to be assessed. In an embodiment, the input module 502 may be a medium, a carrier, a set of mediums, or a set of carries that can hold the saliva sample and the dental plaque sample individually.

The one or more hardware processors 506 are configured to analyze the saliva sample and the dental plaque sample present in the input module 502, using the one or more steps of the method. In an embodiment, the one or more hardware processors 506 are equivalent or same that of the one or more hardware processors 106 of the system 100. The output module 506 is used for displaying the risk assessment of autism spectrum disorder of the subject, based on the analysis of the one or more hardware processors 506. In other words, the output module 506 is used for indicating on the presence or non-presence of the ASD of the subject. In an embodiment, the output module 506 includes but are not limited to a display device, an indicator, a color indicator, or any other equipment that can show the result representation on the ASD to the subject.

The embodiments of the present disclosure provides a mechanism for identifying the risk assessment of autism spectrum disorder of the subject by making use of paired oral microbial samples of both the saliva sample and the dental plaque sample. The present disclosure determines minimum number of microbes, or OTUs or taxonomies for determining the microbial quantitative abundance using which the risk assessment of the subject for the ASD is identified. More specifically, only the five microbes, or OTUs or taxonomies (Mogibacterium, Peptostreptococcus, Eubacterium, Solobacterium, Actinomyces, and Alistipes) of the saliva sample, and the only the five microbes, or OTUs or taxonomies (Eubacterium, Dialister, Atopobium, Enterococcus, Mogibacterium, and Anaeroglobus) of the saliva sample, and in total only the 10 OTUs or taxonomies that are more influenced are identified for determining the microbial quantitative abundance. Hence, the present disclosure requires less resources, simple and yet effective.

The present disclosure is not based on psychiatric analysis and behavioral checklists. It is rather a systematically designed, exhaustively tested and repeatedly validated quantitative methodology for ASD risk assessment and guided therapeutic development.

The present disclosure focuses on oral microbiome of ASD subjects, an avenue which is sparsely touched upon. Dependence on a large number of microbial features for developing the assessment procedure is neither easily translatable nor economical. Moreover, it is difficult to arrive at a relevant and relatable therapeutic solution based on such a large number of microbes in a diagnostic marker. The present disclosure relies on creation of co-related hybrid features (OTU) for each subject through paired sampling. And the disclosed biomarker model is constituted by as less as 10 OTUs taken together from both the saliva sample (5 OTUs) and the dental plaque ample (5 OTUs) microbiome samples with an average AUC of 0.88±0.10 with models giving a held-out AUC of as high as 0.98 for ensemble model developed using hybrid feature set. In hybrid feature based models, the OTUs are not independently considered (unlike the competitive literature) but combined together to create hybrid biomarkers constituted by site-specific microbes. Even site-specific biomarkers are consistently efficient as well as sparse/frugal through the present disclosure. The data splits employed are different which are expected to yield differences in performance, however the smaller number of features with good average cross validated performance is a clear differentiator.

By focusing on frugal combination of disease biomarkers, the present disclosure enables accurate and easy diagnosis of a disease. Importantly, selection of a small set of OTUs (total 10 OTUs taken together from both the saliva sample and the dental plaque sites in a single hybrid ensemble model) as biomarker component. The present disclosure further enables an easy trace-back towards potential causality and guides in focused personalized recommendation design. A systematic diagnostic and personalized recommendation design, with a small set of features, makes it convenient and economical to deploy for mass adoption.

The disclosure provides biomarkers using the described methods employed on the public dataset provided in NCBI SRA database study ID SRP097646. The described methodology can be adopted for other datasets to arrive at additional region/age or other metadata specific models and recommendations.

### Example Scenario

A. Model training: The ML model along with the set of ensemble models are obtained using the data associated with the respective samples. i.e., saliva samples, or dental plaque samples, or both. The data associated with the respective samples is divided into training data and the test data. The present disclosure accepts data in form of a feature table for multiple observations (or samples) wherein each observation/sample is defined by ‘N’ features (F) which are continuous variables and (N≥1). In case of training data (TR), each of the samples/observations further have a preassigned class/category which is binary in nature (e.g., A or B). In case of test data (TS) or data received during actual deployment of the method, the model(s) built based on training data predicts the class/category of the samples/observations.

B. Model training results: The features (microbes) of the single best model and the ensemble model are analyzed and the features that are most frequently occurred are identified, which are then used as the plurality of predetermined microbes to determine the quantitative abundance in real test cases, as explained at step 206 of the method 200.

Table 2 shows the list of features (microbes) of the ensemble model for the hybrid samples (both saliva and dental plaque samples) along with their occurrences. In table 2, SL mean the microbe is associated saliva sample and PQ mean the microbe is associated with the dental plaque sample.

TABLE 2

| Unique Features in | |
|---|---|
| Ensemble Model | Occurrence |
| SL_Mogibacterium | 10 |
| PQ_Eubacterium | 9 |
| PQ_Dialister | 7 |
| PQ_Atopobium | 3 |
| PQ_Enterococcus | 2 |
| SL_Peptostreptococcus | 2 |
| PQ_Mogibacterium | 2 |
| SL_Eubacterium | 2 |
| SL_Solobacterium | 1 |
| SL_Actinomyces | 1 |
| PQ_Anaeroglobus | 1 |
| SL_Alistipes | 1 |

Table 3 shows the list of features (microbes) of the ensemble model for the saliva sample along with their occurrences.

TABLE 3

| Unique Features in Ensemble Model | Occurrence |
|---|---|
| Alloprevotella | 10 |
| Parvimonas | 7 |
| Peptostreptococcus | 6 |
| Anaeroglobus | 4 |
| Morococcus | 4 |
| Solobacterium | 3 |
| Peptococcus | 2 |
| Allisonella | 1 |
| Mesocricetibacter | 1 |
| Anaerovorax | 1 |

Table 4 shows the list of features (microbes) of the ensemble model for the dental plaque sample along with their occurrences.

TABLE 4

| Unique Features in Ensemble Model | Occurrence |
|---|---|
| Mogibacterium | 10 |
| Mesocricetibacter | 10 |
| Catonella | 5 |
| Peptostreptococcus | 5 |
| Anaerovorax | 5 |
| Solobacterium | 4 |
| Parvimonas | 2 |

Table 5 shows an exemplary model metrics data of the single best ML model and the ensemble model that has performed the best for the hybrid model using both saliva sample and dental plaque sample.

TABLE 5

| | Single best | Ensemble |
|---|---|---|
| Model Metrics | ML Model | ML model |
| CV (100 | 0.85 | 0.8792 |
| iterations) | | |
| Mean AUC | | |
| AUC Min-Max | 0.60-1.00 | 0.64-1.00 |
| AUC Std. Dev | 0.110104 | 0.095976 |
| CV (100 | 0.516945 | 0.580859 |
| iterations) | | |
| Mean MCC | | |
| MCC Min-Max | 0.00-1.00 | 0.00-1.00 |
| MCC Std. Dev | 0.258161 | 0.24416 |
| Training AUC | 0.95619 | 0.958095 |
| Training MCC | 0.741941 | 0.826645 |

C. Case Study: A case study is conducted on a subject for whom the risk of ASD to be ascertained and the steps 1 to 8 are mentioned to explain the case study and the steps are in line with the steps of the method 200 of the present disclosure.

At step 1, both the saliva sample and dental plaque samples are collected at the same, as test samples from the subject for whom the risk of ASD to be ascertained.

At step 2, the raw abundances of various microbial taxonomic groups present in the collected samples are quantified and a unified table is created. Methodology used in this step involves extraction of microbial DNA contents from the collected samples followed by amplification and sequencing of either full-length or specific variable regions of the bacterial 16S rRNA marker genes using the next-generation sequencing platform or by using the multiplexed qPCR-based quantification methodology. Table 6 shows the raw abundance of various microbial taxonomic groups present in the collected samples. In Table 6, PQ refers to microbial taxonomic groups related to dental plaque sample and SL refers to microbial taxonomic groups related to saliva sample.

TABLE 6

| Microbial taxonomic groups | Raw abundance |
|---|---|
| PQ_Neisseria | 112 |
| PQ_Morococcus | 23 |
| PQ_Kingella | 46 |
| PQ_Eikenella | 5 |
| . . . | . . . |
| . . . | . . . |
| SL_Neisseria | 1854 |
| SL_Morococcus | 107 |
| SL_Kingella | 15 |

At step 3, the percent normalized abundances values of various microbial taxonomic groups are calculated individually for each of the saliva sample and the dental plaque sample, using the corresponding raw abundances mentioned in Table 6. Table 7 shows the percent normalized abundances values of various microbial taxonomic groups present in the collected samples.

TABLE 7

| Microbial taxonomic groups | Percent normalized abundances values |
|---|---|
| PQ_Neisseria | 1.11813 |
| PQ_Morococcus | 0.214436 |
| PQ_Kingella | 0.459506 |
| PQ_Eikenella | 0.053609 |
| . . . | . . . |
| . . . | . . . |
| SL_Neisseria | 18.5459 |
| SL_Morococcus | 1.07919 |
| SL_Kingella | 0.150666 |
| SL_Eikenella | 0.007008 |

At step 4, From the normalized abundance table, abundances of only the subset of microbial taxonomic groups which overlap with the list of two microbial taxonomic groups that are provided against the Single best training model are retained. Table 8 shows the model characteristics of features in the single best training model.

TABLE 8

| | SL_Mogibacterium | PQ_Dialister | SL_Actinomyces | PQ_Tannerella | SL_Solobacterium | SL_Eubacterium |
|---|---|---|---|---|---|---|
| Q1 | 0 | 0.00182 | 0.777965 | 0.117214 | 0.008376 | 0.005911 |
| Q3 | 0.02678 | 0.0997 | 2.103827 | 0.644688 | 0.078859 | 0.093459 |
| Q2_{A} | 0.028023 | 0.12677 | 1.95256 | 0.645844 | 0.071636 | 0.093373 |
| Q2_{B} | 0.00147 | 0.00268 | 0.890287 | 0.182163 | 0.010563 | 0.00832 |
| Min Model | 0.170987 | | | | | |
| Score | | | | | | |
| Max Model | 0.495151 | | | | | |
| Score | | | | | | |
| Threshold | 0.264753 | | | | | |
| Numerator/ | Denominator | Denominator | Denominator | Denominator | Denominator | Denominator |
| Denominator | | | | | | |

| | |
|---|---|
| Model Type | Reverse |

As an example, assume that the three taxa in the taxonomic abundance profile obtained by processing the hybrid (Saliva, Plaque) sample combination (in the manner mentioned in Steps 1 and 2) had the following rarefied abundances:
Abundance of SL_Actinomyces (i.e., feature 3 in training model) in collected saliva sample: 1.37
Abundance of PQ_Tannerella (i.e., feature 4 in training model) in collected dental plaque sample: 0.03

At step 5, Using Q1 and Q3 values corresponding to each training model feature in the single best training model, and the transformation is applied to the above rarefied abundances. Following are the calculated transformed abundancies:
Transformed abundance (Fg_SL_Actinomyces): 0.357952
Transformed abundance (F_{g}_PQ_Tannerella): 0.0

The transformed abundance of individual features as obtained above are then used appropriately in the candidate model equation (CM_{K}) (as replicated below), and numerator and denominator sums are computed. In this case, the values obtained are as follows—

Since Numerator sum=0 and Denominator sum=1.357952 in this case, a value of 1 is added to both numerator and denominator as per the rules:

$${CM}_{K} = \frac{Σ {F}_{numerator}}{Σ {F}_{denominator}} when {F}_{numerator} > 0 and {F}_{denominator} > 0 or , \\ {CM}_{K} = \frac{Σ {F}_{numerator} + 1}{Σ {F}_{denominator} + 1} when either {F}_{numerator} or {F}_{denominator} = 0$$

Numerator sum: 1.000
Denominator sum: 1.357952

At step 6, the sample model score (MS) is computed using above Numerator sum and Denominator sum. The sample model score (MS) is then transformed into scaled model score (SMS) (having values between −1 and +1, using following rules—

$$SMS = ( MS - {T}_{max} ) / ( {CMS}_{{K}_{max}} - {T}_{max} ) , when MS >= {T}_{max} , and \\ SMS = ( MS - {T}_{max} ) / ( {T}_{max} - {CMS}_{{K}_{min}} ) , when MS < {T}_{max} ,$$

Wherein, Tₘₐₓ, CMS_{K}_{ₘₐₓ}, and CMS_{K}_{ₘᵢₙ} values corresponding to the respective model is used. For this purpose, the values of threshold: 0.264753, a maximum model score: 0.495151, a minimum model score: 0.170987 for single best model (as mentioned in Table 8) are employed.
Model score (MS): 0.736403
Scaled model score (SMS): 0.495151

At step 7, the SMS is then used for predicting the risk of ASD of the individual from whom the saliva sample and the dental plaque samples are obtained. Since both forward model and reverse model are evaluated, the final selected model is then used for classification or prediction). Here in this case, final selected single best model is a reverse model, hence the final prediction score value is calculated as (SMS*−1)
Final pred_score is −0.495151
Since the value is <0, the prediction class is “A” i.e., “Low risk or healthy category”
   Following the same series of steps, if the value of SMS is greater than ‘0’ then prediction class will be “B” and thus the risk category for the subject will be “High risk of ASD”.

At step 8, similarly, for the ensemble ML model, all the steps are repeated for all the single models in the ensemble and finally the average of all the final prediction score is calculated using sample model scores (SMS) and the class prediction is done based on final average prediction score obtained for that sample.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein addresses unresolved problem for accurate identification and selection of subjects with increased risk of the ASD and providing a personalized microbial cocktail for treating the ASD, using oral microbiome of the ASD affected subjects. The present disclosure attempts to offer an early, easy and reliable risk assessment and a method for design of personalized recommendable composition for applications towards amelioration of ASD.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g., any kind of computer like a server or a personal computer, or the like, or any combination thereof. The device may also include means which could be e.g., hardware means like e.g., an application-specific integrated circuit (ASIC), a field-programmable gate array (FPGA), or a combination of hardware and software means, e.g., an ASIC and an FPGA, or at least one microprocessor and at least one memory with software processing components located therein. Thus, the means can include both hardware means, and software means. The method embodiments described herein could be implemented in hardware and software. The device may also include software means. Alternatively, the embodiments may be implemented on different hardware devices, e.g., using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A method for risk assessment of autism spectrum disorder in a subject, comprising the steps of:
collecting a saliva sample and a dental plaque sample of the subject whose risk of autism spectrum disorder is to be assessed;
extracting microbial deoxyribonucleic acid (DNA) sequences from each of the saliva sample and the dental plaque sample, individually;
determining a quantitative abundance of: (i) each of a plurality of predetermined microbes associated with the saliva sample and (ii) each of a plurality of predetermined microbes associated with the dental plaque sample, individually, from respective extracted DNA sequences, using a first set of probes and a second set of probes specific to each of the plurality of predetermined microbes associated with the saliva sample and the dental plaque sample respectively, through a multiplexed quantitative Polymerase Chain Reaction (qPCR) technique;
collating, via one or more hardware processors, the quantitative abundance of: (i) each of the plurality of predetermined microbes associated with the saliva sample and (ii) each of the plurality of predetermined microbes associated with the dental plaque sample, to obtain a hybrid abundance matrix;
determining, via the one or more hardware processors, a model score based on the hybrid abundance matrix, using a pre-determined machine learning (ML) model; and
performing, via the one or more hardware processors, risk assessment of autism spectrum disorder of the subject, based on the model score and a predefined threshold value.

2. The method of claim 1, further comprising:
designing, a personalized recommendation for the subject assessed as having autism spectrum disorder, by utilizing a set of rules for the set of microbes that constitute the pre-determined machine learning model to identify one or more personalized probiotic and antibiotic candidates that ameliorate disease symptoms in the subject identified as having autism spectrum disorder.

3. The method of claim 1, wherein:
(i) the plurality of predetermined microbes associated with the saliva sample comprises of Mogibacterium, Peptostreptococcus, Eubacterium, Solobacterium, Actinomyces, and Alistipes; and
(ii) the plurality of predetermined microbes associated with the dental plaque sample comprises of Eubacterium, Dialister, Atopobium, Enterococcus, Mogibacterium, and Anaeroglobus.

4. The method of claim 1, wherein the first set of probes specific to each of the plurality of predetermined microbes associated with the saliva sample are utilized in a first multiplexed qPCR run, and a second multiplexed qPCR run, to determine the quantitative abundance of each of the plurality of predetermined microbes associated with the saliva sample, and wherein:
(i) the plurality of predetermined microbes, the quantitative abundance of which are being determined through the first multiplexed qPCR run are: Mogibacterium, Peptostreptococcus, Eubacterium, and Solobacterium; and
(ii) the plurality of predetermined microbes, the quantitative abundance of which are being determined through the second multiplexed qPCR run are: Mogibacterium, Peptostreptococcus, Actinomyces, and Alistipes.

5. The method of claim 1, wherein the second set of probes specific to each of the plurality of predetermined microbes associated with the dental plaque sample are utilized in a third multiplexed qPCR run, and a fourth multiplexed qPCR run, to determine the quantitative abundance of each of the plurality of predetermined microbes associated with the dental plaque sample, and wherein:
(i) the plurality of predetermined microbes, the quantitative abundance of which are being determined through the third multiplexed qPCR run are: Eubacterium, Dialister, Atopobium, and Enterococcus; and
(ii) the plurality of predetermined microbes, the quantitative abundance of which are being determined through the fourth multiplexed qPCR run are: Eubacterium, Dialister, Mogibacterium, and Anaeroglobus.

6. The method of claim 1, wherein the pre-determined machine learning (ML) model is an ensemble ML model that is built using a microbial abundance data corresponding to a plurality of training saliva samples and a plurality of training dental plaque samples.

7. The method of claim 1, wherein the plurality of predetermined microbes associated with the saliva sample and the plurality of predetermined microbes associated with the dental plaque sample are features of the pre-determined machine learning (ML) model.

8. The method of claim 4, wherein one or more predetermined microbes out of the plurality of predetermined microbes associated with the saliva sample, are common to the first multiplexed qPCR run and the second multiplexed qPCR run for determining the quantitative abundance, and wherein the one or more predetermined microbes that are common to the first multiplexed qPCR run and the second multiplexed qPCR run are determined based on (i) a median abundance of each of the plurality of predetermined microbes obtained from the plurality of training saliva samples, (ii) a frequency of occurrence of each of the plurality of predetermined microbes constituting the ensemble ML model.

9. The method of claim 5, wherein one or more predetermined microbes out of the plurality of predetermined microbes associated with the dental plaque sample are common to the third multiplexed qPCR run and the fourth multiplexed qPCR run for determining the quantitative abundance, and wherein the one or more predetermined microbes that are common to the third multiplexed qPCR run and the fourth multiplexed qPCR run are determined based on (i) a median abundance of each of the plurality of predetermined microbes obtained from the plurality of training dental plaque samples, (ii) a frequency of occurrence of each of the plurality of predetermined microbes constituting the ensemble ML model.

10. A kit for risk assessment of autism spectrum disorder in a subject, comprising:
an input module for receiving a saliva sample and a dental plaque sample of the subject whose risk of autism spectrum disorder is to be assessed;
one or more hardware processors configured to analyze the saliva sample and the dental plaque sample using the method performed in any of the claim 1 to claim 9; and
an output module for displaying the risk assessment of autism spectrum disorder of the subject, based on the analysis of the one or more hardware processors.
